SEQUENCE LISTING (13 sequences)

SEQ ID NO: 1 - DNA, 195 bp, Artificial Sequence
  LAMP product
gctcattgattctacatattatctttactctttctaattcaaatgtgtttagatatctatatattatcccaaaagtaaaatatcaagtactattcttaatttaatataatttattttattgctattcagattataaatcgaagataaaaaatagcaaaggtgtaataacgactagttccaaaataaaggaaggtc

SEQ ID NO: 2 - DNA, 56 bp, Artificial Sequence
  Ti-FIP  (Tilletia indica)
gaatagtacttgatattttacttttggggctcattgattctacatattatctttac

SEQ ID NO: 3 - DNA, 51 bp, Artificial Sequence
  Ti-BIP  Tilletia indica
gctattcagattataaatcgaagataaagaccttcctttattttggaacta

SEQ ID NO: 4 - DNA, 28 bp, Artificial Sequence
  Ti-LF  Tilletia indica
gatatctaaacacatttgaattagaaag

SEQ ID NO: 5 - DNA, 22 bp, Artificial Sequence
  Ti-LB  Tilletia indica
aatagcaaaggtgtaataacga

SEQ ID NO: 6 - DNA, 25 bp, Artificial Sequence
  Ti-F3  Tilletia indica
ccaaactacgatatagcaatatatc

SEQ ID NO: 7 - DNA, 28 bp, Artificial Sequence
  Ti-B3  Tilletia indica
ttatagttactaagtgtatttgatgttc

SEQ ID NO: 8 - DNA, 21 bp, Artificial Sequence
  Nucleic acid probe
gtgtaataacgactagttcca

SEQ ID NO: 9 - DNA, 21 bp, Artificial Sequence
  Nucleic acid probe
cgaagataaaaatagcaaagg

SEQ ID NO: 10 - DNA, 24 bp, Artificial Sequence
  nucleic acid probe
gatatctatatattatcccaaaag

SEQ ID NO: 11 - DNA, 22 bp, Artificial Sequence
  Forward primer Tilletia indica
ctaattcttttacctgaggtgc

SEQ ID NO: 12 - DNA, 24 bp, Artificial Sequence
  Reverse primer  Tilletia indica
agttactaagtgtatttgatgttc

SEQ ID NO: 13 - DNA, 370 bp, Tilletia indica
ctaattcttttacctgaggtgcctagaggtgatgatacttatataatagattaataataatatactaaattattgttcttaatttagacttcttatatgtatataaaggggggccaaactacgatatagcaatatatctatgctcattgattctacatattatctttactctttctaattcaaatgtgtttagatatctatatattatcccaaaagtaaaatatcaagtactattcttaatttaatataatttattttattgctattcagattataaatcgaagataaaaaatagcaaaggtgtaataacgactagttccaaaataaaggaaggtctttaaaaaaagaacatcaaatacacttagtaact

(19) United States
(12) Patent Application Publication
(10) Pub. No.: US 2020/0131590 A1
(43) Pub. Date: Apr. 30, 2020

(54) Methods, reagents and kits for detection of Karnal bunt

(71) Applicant: The Crown in right of the State of New South Wales acting through the Department of Primary Industri, New South Wales (AU)

(72) Inventor: Mui-Keng Tan, New South Wales (AU)

(21) Appl. No.: 16/666,675

(22) Filed: Oct. 29, 2019

(30) Foreign Application Priority Data
Oct. 29, 2018 (AU) 2018904101

(51) Int. Cl. C12Q 1/6895; A01N 1/02; G01N 33/543

(52) U.S. Cl. CPC C12Q 1/6895; A01N 1/0221; G01N 2333/37; G01N 33/54346; G01N 33/5436

(57) ABSTRACT

The present disclosure relates to methods and test kits for detecting Tilletia indica (Karnal bunt) in biological samples. In particular, the present disclosure relates to a diagnostic method employing isothermal amplification of nucleic acids, namely a loop-mediated amplification (LAMP), in combination with lateral flow technology to detect Karnal bunt in a grain sample, e.g., from wheat or triticale, as well as test kits and reagent mixtures for use in same.

### RELATED APPLICATION DATA

This application claims the right of priority to Australian provisional patent application No. 2018904101, filed on 29 Oct. 2018, the complete contents of which is incorporated by reference herein in its entirety.

### TECHNICAL FIELD

The present disclosure relates to methods and test kits for detecting Tilletia indica (Karnal bunt) in biological samples. In particular, the present disclosure relates to a diagnostic method employing isothermal amplification of nucleic acids, namely loop-mediated amplification (LAMP), in combination with lateral flow technology to detect Karnal bunt in a grain sample, e.g., from wheat or triticale, as well as test kits and reagent mixtures for use in same.

### BACKGROUND

Karnal bunt is caused by the fungus Tilletia indica, and affects wheat, (including bread and durum wheat) and triticale. The fungus produces masses of powdery spores that discolour the grain and grain products, and creates a fishy smell, making grain unpalatable. Flour made from infected wheat is unfit for human consumption due to its objectionable smell and taste.

Karnal bunt is named after Karnal, India, where the disease was first discovered on a wheat crop in 1931. Since then, the disease has been reported in all major wheat-growing states of India, Pakistan, Afghanistan, Mexico, and certain areas of the Southwestern United States such as New Mexico, Arizona, and parts of Texas.

In an incursion scenario, such as in a country or region where Karnal bunt is not historically known to be present (e.g., Australia), grain trade may be temporarily halted in order to enable analysis of large numbers of samples to be undertaken. This is likely to be necessary to provide trading partners with sufficient evidence to satisfy a declaration of Karnal bunt freedom for wheat growing regions or individual consignments of grain. In this regard, the economic impact of the disease has been rated extremely high (Murray and Brennan (1998) Australas Plant Pathol., 27: 212-225; Stansbury et al., (2002) Phytopathology 92:321-331; Wittwer et al., (2005) Australian Journal of Agricultural and Resource Economics 49:75-89).

In the context of Australia, the wheat industry must be able to ensure that wheat exports are free from Karnal bunt to protect premium markets access. For this reason, the development of a high throughput test for Karnal bunt has been identified as a very high priority by both the National Karnal Bunt Working Group and the Sub-Committee for Plant Health Diagnostics (https://grdc.com.au/research/projects/project?id=4313). It has also been raised as a gap in Australia's preparedness activities by the National Plant Biosecurity Research, Development and Extension (RDE) Strategy Implementation Committee. This is to ensure that Australia's wheat exports are free from Karnal bunt so that market access is protected as World Trade Organization regulations demand pest-free evidence.

The current international detection protocol for karnal bunt endorses a range of identification methods, ranging from morphological identification under a microscope to molecular methods using polymerase chain reaction (PCR)-based methods. The latter protocols require expertise of plant pathologists and/or molecular biologists in addition to expensive scientific instrumentation such as thermal cyclers and real-time PCR machines. This places severe constraints in the implementation of existing Karnal bunt diagnostics in a high throughput scenario, such as during an incursion.

Accordingly, there is a need for improved methodologies and means for detecting Karnal bunt which are more amenable to high-throughput screening and performance in the absence of expensive equipment and/or complicated protocols.

### SUMMARY

The ability to effectively and efficiently screen for Karnal bunt is important to the health of the agriculture industry, particularly in those countries which rely on export and/or import of wheat and/or triticale. The present disclosure is based, at least in part, on the recognition by the inventors that there is a need for an improved means of screening for Karnal bunt. In this regard, the present inventors have developed a test kit and diagnostic method for detecting Tilletia indica or spores thereof which is based on an isothermal amplification assay, namely the loop-mediated isothermal amplification (LAMP) assay developed by Tan et al., (2016) PLoS ONE 11(11):e0166086, albeit modified for use with lateral flow technology. In doing so, the inventors have developed a test kit and diagnostic method which does not require sophisticated or expensive scientific equipment, is more user/operator friendly, and has an additional layer of specificity for Karnal bunt so as to avoid false positive results in the event of non-specific hybridisation and/or amplification. The diagnostic test kit and method of the disclosure may improve high-throughput screening capabilities for Karnal bunt, such as may be required in an incursion scenario, and has the capability of being deployed in a wide range of circumstances and environments. For example, test kit and method may be deployed in plant health diagnostic laboratories, grain processing centres, quarantine stations and farmers for routine screening of grain samples for exports, imported products and agricultural machineries that may potentially have contaminants with karnal bunt e.g. imported fertilizers, animal feeds, agricultural machineries (e.g. harvesters on lease from overseas). The inventors have also developed and tested an embodiment of the test kit which is thermostable and does not require a cold-chain for distribution, making it accessible to markets in less-developed regions.

Accordingly, in one example the disclosure provides a test kit for detecting Tilletia indica or spores thereof in a sample, said test kit comprising:
(1) a reagent mixture comprising reagents configured to amplify a nucleic acid sequence of Tilletia indica by an isothermal amplification assay to produce a biotinylated amplification product comprising a sequence set forth in SEQ ID NO: 1;
(2) a nucleic acid probe comprising a polynucleotide sequence of at least 16 nucleotides in length which is sufficiently complementary to a region of corresponding length within the biotinylated amplification product such that the nucleic acid probe and amplification product are hybridisable, wherein the nucleic acid probe is conjugated to a hapten;
(3) one or more lateral flow test strips comprising
   (a) a label-holding portion comprising a mobilisable capture reagent comprising a detectable label, wherein the mobilisable capture reagent is configured to bind to the hapten conjugated to the nucleic acid probe; and
   (b) a test portion comprising an immobilised capture reagent configured to specifically bind biotin and thereby immobilise biotin to the test portion.

In one example, the reagent mixture comprises: at least one mesophilic enzyme for amplifying nucleic acids under isothermal conditions; isothermal amplification primers specific for Tilletia indica; dinucleotide triphosphates (dNTPs); one or more salts; and a buffer.

The isothermal amplification assay may be any isothermal amplification assay type known in the art for amplifying DNA. In one example, the isothermal amplification assay is selected from loop-mediated isothermal amplification (LAMP), recombinase polymerase amplification (RPA) and helicase-dependent amplification (HDA). For example, the isothermal amplification assay may be a LAMP assay. For example, the isothermal amplification assay may be a RPA assay. For example, the isothermal amplification assay may be a HLA assay.

In accordance with an example in which the isothermal amplification assay is a LAMP assay, the reagent mixture comprises: at least one DNA polymerase enzyme for amplifying nucleic acids under isothermal conditions; LAMP primers specific for Tilletia indica; dinucleotide triphosphates (dNTPs); a magnesium salt; and a buffer.

In one example, the reagent mixture comprises the following LAMP primers:

Ti-FIP comprising the sequence set forth in SEQ ID NO:2 or a sequence which is substantially identical thereto, wherein the sequence of Ti-FIP is conjugated to a biotin;

Ti-BIP comprising the sequence set forth in SEQ ID NO:3 or a sequence which is substantially identical thereto;

Ti-LF comprising the sequence set forth in SEQ ID NO:4 or a sequence which is substantially identical thereto;

Ti-LB comprising the sequence set forth in SEQ ID NO:5 or a sequence which is substantially identical thereto;

Ti-F3 comprising the sequence set forth in SEQ ID NO:6 or a sequence which is substantially identical thereto; and

Ti-B3 comprising the sequence set forth in SEQ ID NO:7 or a sequence which is substantially identical thereto.

In one example, the isothermal amplification assay is a LAMP assay and the reagent mixture comprises the following LAMP primers:

Ti-FIP comprising the sequence set forth in SEQ ID NO:2, wherein the sequence of Ti-FIP is conjugated to a biotin;

Ti-BIP comprising the sequence set forth in SEQ ID NO:3;

Ti-LF comprising the sequence set forth in SEQ ID NO:4;

Ti-LB comprising the sequence set forth in SEQ ID NO:5;

Ti-F3 comprising the sequence set forth in SEQ ID NO:6; and

Ti-B3 comprising the sequence set forth in SEQ ID NO:7.

In one example, the DNA polymerase enzyme is selected from the groups consisting of Bst DNA polymerase, Bsm DNA polymerase, Gst DNA polymerase, SD DNA polymerase and combinations thereof. In one particular example, the DNA polymerase enzyme is Bst DNA polymerase.

In one example, the magnesium salt is MgSO₄. In another example, the magnesium salt is MgCl₂.

In one example, the reagent mixture is thermostable. For example, the reagent mixture may be provided in a dried form e.g., lyophilised.

In one example, the nucleic acid probe is provided in a dried form e.g., lyophilised.

In one example, both the reagent mixture and the nucleic acid probe are each provided in a dried form.

In accordance with an example in which the reagent mixture is lyophilised, the reagent mixture may comprises a cryoprotectant. For example, the cryoprotectant may be a sugar, such as a sugar selected from the group consisting of sucrose, trehalose, glucose, galactose, maltose, mannitol, lactose and derivatives thereof. For example, the cryoprotectant may be selected from sucrose and trehalose. In one example, the cryoprotectant is sucrose. In one example, the cryoprotectant is trehalose.

The cryoprotectant may be present in an amount of about 7% w/v to about 8% w/v. In one example, the cryoprotectant is sucrose which is present in the reagent mixture at about 7.5% w/v. In another example, the cryoprotectant is trehalose which is present in the reagent mixture at about 7.5% w/v.

In another example, the reagent mixture is provided in a liquid or frozen form and the nucleic acid probe is provided in a dried form.

In one example, the nucleic acid probe is substantially identical to a polynucleotide sequence set forth in any one of SEQ ID NOs: 8-10.

In one example, the nucleic acid probe is at least 16 nucleotides in length and is substantially identical to a region of corresponding length within the polynucleotide sequence set forth in SEQ ID NO:8. In one example, the nucleic acid probe is at least 17 nucleotides in length and is substantially identical to a region of corresponding length within the polynucleotide sequence set forth in SEQ ID NO:8. In one example, the nucleic acid probe is at least 18 nucleotides in length and is substantially identical to a region of corresponding length within the polynucleotide sequence set forth in SEQ ID NO:8. In one example, the nucleic acid probe is at least 19 nucleotides in length and is substantially identical to a region of corresponding length within the polynucleotide sequence set forth in SEQ ID NO:8. In one example, the nucleic acid probe is at least 20 nucleotides in length and is substantially identical to a region of corresponding length within the polynucleotide sequence set forth in SEQ ID NO:8. In one example, the nucleic acid probe is substantially identical to the polynucleotide sequence set forth in SEQ ID NO:8. For example, the nucleic acid probe may comprise the polynucleotide sequence set forth in SEQ ID NO: 8.

In one example, the nucleic acid probe is at least 16 nucleotides in length and is substantially identical to a region of corresponding length within the polynucleotide sequence set forth in SEQ ID NO:9. In one example, the nucleic acid probe is at least 17 nucleotides in length and is substantially identical to a region of corresponding length within the polynucleotide sequence set forth in SEQ ID NO:9. In one example, the nucleic acid probe is at least 18 nucleotides in length and is substantially identical to a region of corresponding length within the polynucleotide sequence set forth in SEQ ID NO:9. In one example, the nucleic acid probe is at least 19 nucleotides in length and is substantially identical to a region of corresponding length within the polynucleotide sequence set forth in SEQ ID NO:9. In one example, the nucleic acid probe is at least 20 nucleotides in length and is substantially identical to a region of corresponding length within the polynucleotide sequence set forth in SEQ ID NO:9. In one example, the nucleic acid probe is substantially identical to the polynucleotide sequence set forth in SEQ ID NO:9. For example, the nucleic acid probe may comprise the polynucleotide sequence set forth in SEQ ID NO: 9.

In one example, the nucleic acid probe is at least 16 nucleotides in length and is substantially identical to a region of corresponding length within the polynucleotide sequence set forth in SEQ ID NO:10. In one example, the nucleic acid probe is at least 17 nucleotides in length and is substantially identical to a region of corresponding length within the polynucleotide sequence set forth in SEQ ID NO:10. In one example, the nucleic acid probe is at least 18 nucleotides in length and is substantially identical to a region of corresponding length within the polynucleotide sequence set forth in SEQ ID NO:10. In one example, the nucleic acid probe is at least 19 nucleotides in length and is substantially identical to a region of corresponding length within the polynucleotide sequence set forth in SEQ ID NO:10. In one example, the nucleic acid probe is at least 20 nucleotides in length and is substantially identical to a region of corresponding length within the polynucleotide sequence set forth in SEQ ID NO:10. In one example, the nucleic acid probe is at least 21 nucleotides in length and is substantially identical to a region of corresponding length within the polynucleotide sequence set forth in SEQ ID NO:10. In one example, the nucleic acid probe is at least 22 nucleotides in length and is substantially identical to a region of corresponding length within the polynucleotide sequence set forth in SEQ ID NO:10. In one example, the nucleic acid probe is at least 23 nucleotides in length and is substantially identical to a region of corresponding lengthin with the polynucleotide sequence set forth in SEQ ID NO:10. In one example, the nucleic acid probe is substantially identical to the polynucleotide sequence set forth in SEQ ID NO:10. For example, the nucleic acid probe may comprise the polynucleotide sequence set forth in SEQ ID NO: 10.

In one particular example of the test kit of the disclosure:

(a) the reagent mixture is thermostable and comprises:
(i) a Bst DNA polymerase;
(ii) the LAMP primers: Ti-FIP (SEQ ID NO: 2) conjugated to a biotin; Ti-BIP (SEQ ID NO: 3); Ti-LF (SEQ ID NO: 4); Ti-LB (SEQ ID NO: 5); Ti-F3 (SEQ ID NO: 6); and Ti-B3 (SEQ ID NO: 7);
(iii) dNTPs;
(iv) MgSO₄; and
(v) a buffer; and
   (b) the nucleic acid probe comprises the polynucleotide sequence set forth in SEQ ID NO: 8.

In one example, the immobilised capture reagent at the test portion of the lateral test strip is a biotin ligand.

In one example, the mobilisable capture reagent of the label-holding portion of the lateral test strip is an antibody which binds the hapten, and wherein the antibody is conjugated to the detectable label e.g. a gold nanoparticle, latex nanoparticle or a fluorescent quantum dot (QD).

In one example, the hapten is a fluorophore. For example, the hapten may be a fluorophore selected from fluorescein, fluorescein isothiocyanate (FITC) or 6-FAM. In one particular example, the hapten is FITC.

In another example, the hapten is a non-fluorescent hapten molecule selected from the group consisting of dinitrophenyl (DNP), digoxygenin, and nitrotyrosine. For example, the hapten may be digoxigenin.

In one example, the one or more lateral flow test strips comprise a control portion comprising an immobilised capture reagent configured to detect the mobilisable capture reagent. For example, the immobilised capture reagent at the control portion may be an antibody against the mobilisable capture reagent. Preferably, the control portion is located downstream of the test portion on the test strip.

In one example, the one or more lateral test strips comprise a sample receiving portion for receiving a liquid sample.

In any of the foregoing examples, the test kit may further comprise an aqueous running buffer for the lateral flow test strip(s).

In any of the foregoing examples, the test kit may further comprise an assay positive control comprising isolated Tilletia indica DNA or a fragment thereof suitable as a template for the isothermal amplification assay e.g., a LAMP assay.

The test kit of the disclosure may comprise an apparatus configured to receive the lateral flow test strip(s) and present information about the identification of Tilletia indica or spores thereof in a sample to a user via a display. The apparatus may be configured to allow removal of a used test strip from its casing after use and subsequent replacement with a new test strip.

In one example, the apparatus is provided in the form of a hand-held device.

In one example, the apparatus may comprise a reader to identify Tilletia indica or spores thereof in the sample. For example, the reader may include one or more photodetectors capable of monitoring light reflection or light output at the test portion.

The present disclosure also provides a method of detecting the presence or absence of Tilletia indica nucleic acids in a sample, said method comprising:
(a) performing an isothermal amplification assay on DNA extracted from the sample (sample DNA) using a reagent mixture of the test kit as described herein;
(b) incubating the product of (a) in the presence of a nucleic acid probe of the test kit as described herein;
(c) contacting the product of (b) with the sample receiving portion of a test strip of the test kit as described herein;
(d) detecting the presence or absence of an amplification product at the test portion of the test strip; and
(e) determining the presence or absence of Tilletia indica nucleic acids in the sample based on the presence or absence of an amplification product at the test portion.

In one example, detecting a signal at the test portion is indicative of the presence of Tilletia indica nucleic acids in the sample and detecting no signal at the test portion is indicative of the absence of Tilletia indica nucleic acids in the sample.

In one example, detecting a signal at the control portion of the lateral flow test strip is indicative that the sample has flowed through the test strip.

In one example, a volume of buffer is added to the sample following the incubation at (b) and prior to contacting the sample with the sample receiving portion of the test strip.

In any of the foregoing examples, the method may comprise the step of isolating DNA from the sample prior to step (a).

In one example, the reagent mixture is provided in a dried form and the method comprises adding a suitable diluent e.g., water, to the reagent mixture at (a). In accordance with this example, the reagent mixture may be thermostable.

In one example, the isothermal amplification assay is performed at about 60° C. to about 66° C. For example, the isothermal amplification assay may be performed at 64° C.

In one example, the incubation step at (b) is performed at about the same temperature as the isothermal amplification assay. For example, the incubation step at (b) and the isothermal amplification assay are both performed at about 64° C.

The isothermal amplification assay may be any isothermal amplification assay type known in the art for amplifying DNA. In one example, the isothermal amplification assay is selected from a LAMP assay, a RPA assay and a HLA assay. In one example, the isothermal amplification assay is a LAMP assay as described herein.

In one example, the sample to be tested using the method or kit of the disclosure is grain or a part thereof, a commodity comprising grain or a part thereof, or debris comprising grain or a part thereof as a contaminant. In other examples, grain or a part thereof is washed with a solution (e.g., an aqueous solution) and the sample to be tested is debris collected from the solution post wash (e.g., using a filter or sieve). In accordance with this example, the debris collected from the solution may comprise one or more spores from Tilletia indica.

In each of the foregoing examples describing a sample suitable for testing with the method or kit of the disclosure, the grain may be from wheat or triticale.

### BRIEF DESCRIPTION OF DRAWINGS

Embodiments of the present disclosure will now be described by way of example with reference to the accompanying drawings, in which:

FIG. 1 shows a top view configuration of the test strip according to one embodiment of the present disclosure;

FIG. 2 shows an oblique view of a hand-held test device according to one embodiment of the present disclosure which may be used in conjunction with the test strip of FIG. 1;

FIG. 3 shows a cross-sectional view of the test device of FIG. 2 along line A-A of FIG. 2;

FIG. 4 shows a schematic representation of reading apparatus used in the test device of FIGS. 2 and 3;

FIG. 5 shows (A) a schematic representation of the diagnostic method of the disclosure using the test kit of the disclosure, and (B) the results of the diagnostic method performed in Example 1 using the test kit of the disclosure;

FIG. 6 shows an agarose gel resolving the products of the LAMP assays performed in Examples 2 and 3; and

FIG. 7 shows an agarose gel resolving the products of the LAMP assays performed in Example 4.

### KEY TO THE SEQUENCE LISTING

SEQ ID NO: 1 Provides a DNA sequence of the LAMP product produced by the LAMP assay of the disclosure.
SEQ ID NO: 2 Provides the DNA sequence of the LAMP primer designated Ti-FIP in the reagent mixture.
SEQ ID NO: 3 Provides the DNA sequence of the LAMP primer designated Ti-BIP in the reagent mixture.
SEQ ID NO: 4 Provides the DNA sequence of the LAMP primer designated Ti-LF in the reagent mixture.
SEQ ID NO: 5 Provides the DNA sequence of the LAMP primer designated Ti-LB in the reagent mixture.
SEQ ID NO: 6 Provides the DNA sequence of the LAMP primer designated Ti-F3 in the reagent mixture.
SEQ ID NO: 7 Provides the DNA sequence of the LAMP primer designated Ti-B3 in the reagent mixture.
SEQ ID NO: 8 Provides the DNA sequence of the nucleic acid hybridisation probe in the test kit.
SEQ ID NO: 9 Provides the DNA sequence of nucleic acid hybridisation probe alternative 1 for inclusion in a test kit of the disclosure.
SEQ ID NO: 10 Provides the DNA sequence of nucleic acid hybridisation probe alternative 2 for inclusion in a test kit of the disclosure.
SEQ ID NO: 11 Provides the DNA sequence of the forward primer used to amplify the Tilletia indica positive DNA control in the test kit.
SEQ ID NO: 12 Provides the DNA sequence of the reverse primer used to amplify the Tilletia indica positive DNA control in the test kit.
SEQ ID NO: 13 Provides the DNA sequence of the Tilletia indica positive DNA control in the test kit.

### DETAILED DESCRIPTION

### General

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example or embodiment of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Selected Definitions

The term “isothermal amplification” in the context of the present disclosure refers to a nucleic acid amplification reaction which takes place at a temperature that does not significantly change during the reaction. In one example, the temperature of the isothermal amplification reaction does not deviate by more than 10° C., preferably by not more than 5° C., even more preferably not more than 2° C. during the enzymatic reaction step(s) where amplification takes place. Accordingly, an “isothermal amplification assay” will be understood to mean a nucleic acid amplification assay which is capable of isothermal amplification of a nucleic acid template (e.g., DNA) at a temperature that does not significantly change during the amplification reaction. Any isothermal amplification method known in the art is contemplated herein. Exemplary isothermal amplification methods which may be used in accordance with the present disclosure include loop-mediated isothermal amplification (LAMP) (Notomi et al. (2000) Nucleic Acids Res 28(12):e63), recombinase polymerase amplification (RPA) (Piepenburg et al. (2006) PloS Biol 4(7):1115-11.20) and helicase-dependent amplification (HDA) (Vincent et al. (2004) EMBO rep 5(8):795-800). However, it will be appreciated that other isothermal amplification assays exist and may be used in the method or with the device of the disclosure. The choice of assay will depend on the type of nucleic acid to be amplified. (e.g., DNA or RNA). Other isothermal amplification assays include, for example, thermostable HDA (tHDA) (An et al. (2005) J Biol Chem 280(32):28952-28958), strand displacement amplification (SDA) (Walker et al. (1992) Nucleic Acids Res 20(7):1691-6), multiple displacement amplification (MDA) (Dean et al. (2002) Proc Natl Acad Sci USA 99(8): 5261-5266), rolling-circle amplification (RCA) (Liu et al. (1996) J Am Chem Soc H8:1587-1594), restriction aided RCA (Wang et al. (2004) Genome Res 14:2357-2366), single primer isothermal amplification (SPIA) (Daffom et al. (2004) Biotechniques 37(5):854-7), transcription mediated amplification (TMA) (Vuorinen et al. (1995) J Clin Microbiol 33: 1856-1859), nicking enzyme amplification reaction (NEAR) (Maples et al. US2009017453), exponential amplification reaction (EXPAR) (Van Ness et al (2003) Proc Natl Acad Sci USA 100(8):4504-4509), nucleic acid sequence based amplification (NASBA) (Kievits et al. (1991) J Virol Methods 35:273-286), and smart-amplification process (SMAP) (Mitani et al. (2007) Nat Methods 4(3):257-62).

Depending on the method of isothermal amplification used different enzymes are required for the amplification reaction. Known isothermal methods for amplification of nucleic acids are mentioned above, wherein the at least one mesophilic enzyme for amplifying nucleic acids under isothermal conditions is selected from the group consisting of mesophilic polymerases, mesophilic polymerases having strand displacement activity, helicase, nicking enzymes, recombination proteins, ligases, glycosylases and/or nucleases.

As used herein, the term “amplification product” shall be understood to mean the DNA amplification product resulting from an isothermal amplification assay performed in accordance with the method or protocol of the disclosure. According to an example in which a LAMP assay is used, the amplification product will be understood to be a “LAMP product”. Similarly, where a HDA or RPA assay is used, the amplification product would be referred to as a HDA product or RPA product respectively. A LAMP product typically comprises a series of concatemers to a target region, which gives rise to a characteristic “ladder” or banding pattern when resolved on a gel.

As used herein, the term “nucleic acid probe” refers to a nucleic acid molecule i.e., a polynucleotide sequence, having a DNA sequence which is substantially complementary to a target nucleic acid sequence and capable of hybridizing to the target nucleic acid to form a duplex. The sequence of the probe may be completely or substantially complementary to the target nucleic acid sequence i.e., include a number of mismatches, provided that the probe has sufficient complementarity to the cognate target sequence to permit hybridisation thereto.

As used herein, the terms “substantial complementarity”, “substantially complementary” and “sufficiently complementary” as used in the context of primers and probes is intended to indicate a sufficient degree of complementarity or precise pairing such that stable and specific binding occurs between nucleic acid sequences e.g., between a nucleic acid probe or primer and the target sequence. It is understood that the sequence of a primer or probe need not be 100% complementary to that of its target sequence. The term encompasses a sequence complementary to another sequence with the exception of mismatches which do not prevent hybridisation and subsequent formation of a duplex. In some cases, the primer or probe is complementary to the target sequence with the exception of 1-2 mismatches. In some cases, the sequences are complementary except for 1 mismatch. In some cases, the sequences are complementary except for 2 mismatches. In other cases, the sequences are complementary except for 3 mismatches. In yet other cases, the sequences are complementary except for 4 mismatches. In the case of primers it is preferred that the penultimate base at the 3′ end of the primer is able to base pair with the template nucleic acid to permit elongation (discussed below for the term “primer”).

As used herein, the term “primer” or similar refers to an enzymatically extendable oligonucleotide that comprises a defined sequence that is designed to hybridize in an antiparallel manner with a complementary, primer-specific portion of a target nucleic acid sequence. Thus, the primer, which is generally provided in molar excess relative to its target polynucleotide sequence, primes template-dependent enzymatic DNA synthesis and amplification of the target sequence. A primer nucleic acid does not need to have 100% complementarity with its template subsequence for primer elongation to occur; primers with less than 100% complementarity can be sufficient for hybridization and enzymatic (e.g., polymerase) elongation to occur provided that there is sufficient complementarity for hybridisation to the target sequence and the penultimate base at the 3′ end of the primer is able to base pair with the template nucleic acid. A primer is preferably, but not necessarily, synthetic, and will generally be about 10 to about 100 nucleotides in length.

The term “substantial identity”, “substantially identical” or similar, as used herein in the context of primers and probes of the disclosure, is intended to indicate that, when optimally aligned with a reference sequence (e.g., as specified by reference to one or more SEQ ID NOs), there is nucleotide sequence identity in at least about 85%, and more preferably at least about 90%, 95%, 96%, 97%, 98% or 99%, of the nucleotide bases in the respective sequences, as measured by any well-known algorithm of sequence identity, such as FASTA, BLAST or Gap.

### Test Kits

The present disclosure provides a test kit for detecting Tilletia indica or spores thereof in a sample. As described herein, the test kit of the disclosure is based on an isothermal amplification assay for amplifying a target region within the Tilletia indica genome e.g., such as the loop-mediated isothermal amplification (LAMP) assay developed by Tan et al., (2016) PLoS ONE 11(11):e0166086, albeit modified so as to not require sophisticated (and often expensive) scientific equipment, to be more operator friendly than a standard DNA amplification assay, and to provide an additional layer of specificity for Karnal bunt so as to avoid false positive results in the event of non-specific hybridisation.

Accordingly, in one example the present disclosure provides a test kit for detecting Tilletia indica or spores thereof in a sample, wherein a test kit comprises:
(1) a reagent mixture comprising reagents configured to amplify a nucleic acid sequence of Tilletia indica by an isothermal amplification assay and thereby produce a biotinylated amplification product comprising a sequence set forth in SEQ ID NO: 1 or a portion thereof;
(2) a nucleic acid probe comprising a polynucleotide sequence of at least 16 nucleotides in length which is sufficiently complementary to a region of corresponding length within the biotinylated amplification product such that the nucleic acid probe and amplification product are hybridisable, wherein the nucleic acid probe is conjugated to a hapten;
(3) one or more lateral flow test strips comprising
   (a) a label-holding portion comprising a mobilisable capture reagent comprising a detectable label, wherein the mobilisable capture reagent is configured to bind to the hapten conjugated to the nucleic acid probe; and
   (b) a test portion comprising an immobilised capture reagent configured to specifically bind biotin and thereby immobilise biotin to the test portion.

The test kit may also comprise (4) a lateral flow assay (LFA) running buffer. A LFA running buffer is typically used to assist an analyte (in this case an isothermal assay reaction mixture potentially containing an amplification product, such as a LAMP product) to travel through the lateral flow test strip.

The test kit may also comprise (5) one or more isothermal amplification assay positive controls.

The test kit components may be packaged together as separate components. The test kit of the disclosure may further comprise instruction for use. The instructions for use may provide directions for using the test kit to determine whether or not a sample is contaminated by Karnal bunt or spores thereof in accordance with a method of one or more embodiment of the present disclosure.

The various components of the test kit are described in further detail below.

### Reagent Mixtures

As described herein, the test kit comprises a reagent mixture comprising reagents configured to amplify a nucleic acid sequence of Tilletia indica using an isothermal amplification assay and thereby produce a biotinylated amplification product comprising a sequence set forth in SEQ ID NO: 1 or a portion thereof. Exemplary isothermal amplification assays are described herein. However, in one example, the isothermal amplification assay is a LAMP assay. Accordingly, the reagent mixture may comprise at least one DNA polymerase enzyme, LAMP primers specific for Tilletia indica, dinucleotide triphosphates (dNTPs), a magnesium salt, and a buffer.

In one example, the reagent mixture comprises the following LAMP primers:

Ti-FIP comprising the sequence set forth in SEQ ID NO:2 or a sequence which is substantially identical thereto, wherein the sequence of Ti-FIP is conjugated to a biotin;

Ti-BIP comprising the sequence set forth in SEQ ID NO:3 or a sequence which is substantially identical thereto;

Ti-LF comprising the sequence set forth in SEQ ID NO:4 or a sequence which is substantially identical thereto;

Ti-LB comprising the sequence set forth in SEQ ID NO:5 or a sequence which is substantially identical thereto;

Ti-F3 comprising the sequence set forth in SEQ ID NO:6 or a sequence which is substantially identical thereto; and

Ti-B3 comprising the sequence set forth in SEQ ID NO:7 or a sequence which is substantially identical thereto.

In one example, one or more of the LAMP primers Ti-FIP, Ti-BIP, Ti-LF, Ti-LB, Ti-F3 and Ti-B3 may be at least 85% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 90% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 95% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 96% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 97% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 98% identical to a primer sequences set forth in SEQ ID NO:2, SEQ ID NO:3, SEQ ID NO:4, SEQ ID NO:5, SEQ ID NO:6 and SEQ ID NO:7 respectively. In one example, one or more of the LAMP primers Ti-FIP, Ti-BIP, Ti-LF, Ti-LB, Ti-F3 and Ti-B3 may be at least 99% identical to a primer sequences set forth in SEQ ID NO:2, SEQ ID NO:3, SEQ ID NO:4, SEQ ID NO:5, SEQ ID NO:6 and SEQ ID NO:7 respectively. In one example, the LAMP primers Ti-FIP, Ti-BIP, Ti-LF, Ti-LB, Ti-F3 and Ti-B3 are identical to the primer sequences set forth in SEQ ID NO:2, SEQ ID NO:3, SEQ ID NO:4, SEQ ID NO:5, SEQ ID NO:6 and SEQ ID NO:7 respectively.

The final concentration of each primer in the reagent mixture may vary. In one example, the respective primers may each be present at a final concentration in the range of about 0.1 μM to about 204. In one example, each of the primers is present at the same final concentration. In another example, one or more of the primers are present in the reagent mixture at differing final concentrations (relative to the other primers in the reagent mixture). In one particular example, the reagent mixture comprises Ti-FIP-Biotin at a final concentration of about 1.6 μM, Ti-BIP at a final concentration of about 1.6 μM, Ti-LF at a final concentration of about 0.4 μM, Ti-LB at a final concentration of about 0.4 μM, Ti-F3 at a final concentration of about 0.2 μM and Ti-B3 at a final concentration of about 0.2μM.

As described herein, the reagent mixture will comprise one or more mesophilic enzymes for amplifying nucleic acids under isothermal conditions. The choice of enzyme will depend on the choice of isothermal amplification assay used. A skilled person would be able to select an appropriate enzyme based on the choice of isothermal amplification assay. However, in accordance with an example in which the reagent mixture is configured for a LAMP assay, the reagent mixture will comprise one or more DNA polymerase enzymes suitable for use in a LAMP assay. The DNA polymerase enzyme is preferably a thermophilic DNA polymerase enzyme with strong strand displacement activity. For example, the DNA polymerase enzyme may be selected from the group consisting of Bst DNA polymerase, Bsm DNA polymerase, Gst DNA polymerase, SD DNA polymerase and combinations thereof. Other DNA polymerases which may employed in a LAMP assay and included in the reagent mixture described herein will be known to a person skilled in the art. In one particular example, the DNA polymerase present in the reagent mixture is Bst DNA polymerase.

The skilled person will understand that the amount of the mesophilicenzyme for nucleic acid amplification (e.g., DNA polymerase) included in the reagent mixture will depend on the number of reactions to be performed using the reagent mixture. In this regard, the reagent mixture can be scaled to accommodate any number of reactions. However, in one particular example, the reagent mixture comprises about 8 units of DNA polymerase per reaction. However, the skilled person will appreciate that this may be varied depending on the efficiency of the polymerase enzyme and LAMP assay.

As described herein, the reagent mixture will comprise dNTPs, which are the building block for synthesis of new DNA during the LAMP assay. The dNTPs will comprise dATP, dCTP, dGTP, and dTTP. These four dNTPs will preferably be present in equimolar amounts for optimal base incorporation during the LAMP assay. This is typically at a final concentration of about 0.2 mM for each dNTP. However, the relative molar amounts and the final concentrations may be varied as required. For example, higher concentrations of dNTPs may be desired in the presence of high levels of Mg²⁺, since Mg²⁺ binds to dNTPs and reduces their availability for incorporation into the new DNA strand during amplification. However, a skilled person will also appreciate that a concentration of dNTPs which is too high can also inhibit amplification. In one example, the reagent mixture comprises dNTPs at a final concentration of about 0.8 mM to about 2 mM. In one example, the reagent mixture comprises dNTPs at a final concentration of about 1.4 mM.

The reagent mixture may also comprise a magnesium salt. In this regard, magnesium ion (Mg²⁺) functions as a cofactor for activity of DNA polymerase by enabling incorporation of dNTPs during polymerization. The magnesium ions at the enzyme's active site catalyze phosphodiester bond formation between the 3′-OH of a primer and the phosphate group of a dNTP. In addition, Mg²⁺ facilitates formation of the complex between the primers or probe and the DNA template by stabilising negative charges on their phosphate backbones. The magnesium salt may be provided in any suitable form known to a person skilled in the art. For example, the magnesium salt may be magnesium chloride (MgCl₂) or magnesium sulphate (MgSO₄). In one example, the magnesium salt in the reagent mixture is MgSO₄. The concentration of magnesium salt may be optimised according to the conditions of the isothermal amplification assay e.g., by titration. In this regard, too low a Mg²⁺ concentration may result in little or no amplification product, due to the DNA polymerase's reduced activity. On the other hand, too high a Mg²⁺ concentration may result in non-specific amplification products as a result of enhanced stability of primer-template complexes, as well as increases in replication errors from misincorporation of dNTPs. A typical final concentration for Mg²⁺ in a isothermal amplification assay (e.g., LAMP assay) is in the range of 1-10 mM. In one example, the reagent mixture comprise a final concentration of Mg²⁺ of 6 mM.

As described herein, the reagent mixture may also comprise a buffer e.g., an isothermal amplification buffer, to ensure a suitable chemical environment for activity of the mesophilic enzyme (e.g., DNA polymerase) during the isothermal amplification assay. There are a number of buffers known in the art and commercially available for use in nucleic acid amplification assays, and these are contemplated herein. The choice of buffer may depend on the choice of enzyme used for amplification (e.g., the choice of DNA polymerase). For example, where a Bst DNA polymerase is present in the reagent mixture (e.g., Bst 2.0 Warmstart® DNA polymerase), an isothermal amplification buffer suitable for Bst DNA polymerase (e.g., 10× Isothermal Amplification Buffer (Bst 2.0)) may be included in the reagent mixture.

It is contemplated that the test kit may be provided with a reagent mixture which requires storage at freezing temperatures e.g., −20° C. or below. An exemplary reagent mixture of the disclosure which requires storage at freezing temperatures is described in Table 2.

In another example, it is contemplated that the reagent mixture may be a thermostable reagent mixture which is provided in a dried form e.g., lyophilised, and which may be transported at ambient temperatures. In accordance with an example in which the reagent mixture is lyophilised, the reagent mixture may further comprise a cryoprotectant. Suitable cryoprotectants for use in lyophilisation are known in the art and contemplated herein. The cryoprotectant may be a sugar selected from the group consisting of sucrose, trehalose, glucose, galactose, maltose, mannitol, lactose and derivatives thereof. For example, the cryoprotectant may be selected from sucrose and trehalose. In one example, the cryoprotectant is sucrose. In one example, the cryoprotectant is trehalose. The cryoprotectant may be present in an amount of about 7% w/v to about 8% w/v. In one particular example, the cryoprotectant is sucrose which is present in the reagent mixture at about 7.5% w/v. In another particular example, the cryoprotectant is trehalose which is present in the reagent mixture at about 7.5% w/v. An exemplary reagent mixture of the disclosure which is provided in a lyophilised form is described in Table 3.

### Nucleic Acid Probe(s)

As described herein, the test kit of the disclosure comprises a nucleic acid probe comprising a polynucleotide sequence of at least 16 nucleotides in length which is sufficiently complementary to a region of corresponding length within a biotinylated amplification product (i.e., produced by the reagent mixture following the isothermal amplification assay), such that the nucleic acid probe and amplification product are hybridisable, and wherein the nucleic acid probe is conjugated to a hapten. For example, the nucleic acid probe may comprise a polynucleotide sequence of at least 16 nucleotides in length, or at least 17 nucleotides in length, or at least 18 nucleotides in length, or at least 19 nucleotides in length, or at least 20 nucleotides in length, or at least 21 nucleotides in length, or at least 22 nucleotides in length, or at least 23 nucleotides in length, or at least 24 nucleotides in length, which is sufficiently complementary to a region of corresponding length within the biotinylated amplification product such that it may hybridise thereto e.g., at a temperature that does not significantly vary to the temperature of the isothermal amplification assay.

In one example, the reagent mixture is configured to produce an amplification product (e.g., a LAMP product) comprising a polynucleotide having the sequence set forth in SEQ ID NO:1. In accordance with this example, a nucleic acid probe of the disclosure will comprise a polynucleotide sequence which is sufficiently complementary to a region of corresponding length in the sequence set forth in SEQ ID NO:1 to permit hybridisation thereto. For example, the nucleic acid probe may comprise a sequence comprising at least 16 contiguous nucleotides of a sequence set forth in any one of SEQ ID NOs: 8-10 or a sequence substantially identical to any one of those sequences. In one example, the nucleic acid probe is at least 16 nucleotides in length (e.g., at least 16, or 17, or 18, or 19, or 20, or 21 nucleotides in length) and is substantially identical to a region of corresponding length within the polynucleotide sequence set forth in SEQ ID NO:8. In one example, the nucleic acid probe is substantially identical to the polynucleotide sequence set forth in SEQ ID NO:8. For example, the nucleic acid probe may comprise the polynucleotide sequence set forth in SEQ ID NO:8. In another example, the nucleic acid probe is at least 16 nucleotides in length (e.g., at least 16, or 17, or 18, or 19 or 20, or 21 nucleotides in length) and is substantially identical to a region of corresponding length within the polynucleotide sequence set forth in SEQ ID NO:9. In one example, the nucleic acid probe is substantially identical to the polynucleotide sequence set forth in SEQ ID NO:9. For example, the nucleic acid probe may comprise the polynucleotide sequence set forth in SEQ ID NO:9. In another example, the nucleic acid probe is at least 16 nucleotides in length (e.g., at least 16, or 17, or 18, or 19 or 20, or 21, or 22, or 23, or 24 nucleotides in length) and is substantially identical to a region of corresponding length within the polynucleotide sequence set forth in SEQ ID NO:10. In one example, the nucleic acid probe is substantially identical to the polynucleotide sequence set forth in SEQ ID NO:10. For example, the nucleic acid probe may comprise the polynucleotide sequence set forth in SEQ ID NO:10. However, other nucleic acid probe sequences capable of binding to other regions within the sequence set forth in SEQ ID NO:1 may be designed using software available in the art and are contemplated for use herein. Where further probes are designed, they would preferably hybridise to the LAMP product at a temperature that does not vary significantly to the temperature used in the isothermal amplification assay (e.g., the same temperature). The nucleic acid probe may also satisfy one or more, preferably all, of the following criteria:
it must not hybridise to any of the six primers in the reagent mixture at the reaction temperature;
it must have sufficient GC content for specific hybridisation at the same or similar temperature as the isothermal amplification assay (e.g., 64° C. in the case of a LAMP assay);
it must not form a secondary structure strong or significant enough to hamper hybridization to the target region i.e., the amplification product; and
it must not result in the formation of primer dimer.

In one example, the nucleic acid probe is provided in the test kit in a dried form. For example, the nucleic acid probe may be provided in a lyophilised form. However, it is also contemplated that the probe may be dried by other means (as described in the Examples hereof).

As described herein, the nucleic acid probe is conjugated to a hapten. As used herein, the term “hapten” shall be understood to mean a molecule that can be recognised and bound by an antibody capture reagent. A hapten for use in accordance with the present disclosure may be a fluorescent dye or a non-fluorescent molecule. Exemplary fluorescent dyes which may be conjugated to the nucleic acid probe include, but not limited to fluorescein/Oregon Green, fluorescein isothiocyanate (FITC), 6-Carboxyfluorescein, tetramethylrhodamine, Texas Red, dansyl, Alexa Fluor 488, BODIPY FL, lucifer yellow, and Alexa Fluor 405/Cascade Blue fluorophores. Antibodies against these dye molecules are commercially available and will be known to a person skilled in the art. Exemplary non-fluorescent haptens molecules include, but not limited to, dinitrophenyl (DNP), digoxygenin, and nitrotyrosine. In use, the nucleic acid probe will hybridise to the amplification product (e.g., LAMP product) to form a complex (comprised of the nucleic acid probe and the amplification product) which can then be detected using the mobilisable capture reagent labelled with an appropriate detectable label on the lateral flow test strip. The skilled person will appreciate that the choice of hapten may vary depending on the capture reagent used. In one example, the hapten conjugated to the nucleic acid probe is a fluorophore e.g., FITC or 6-FAM. In another example, the hapten is digoxygenin. However, other haptens, including those described herein, are also contemplated.

### Positive Control

As discussed herein, the test kit of the disclosure may also comprise one or more isothermal amplification assay positive controls for use in the method of the disclosure. For example, the isothermal amplification assay positive control may be an isolated Tilletia indica nucleic acid (e.g., DNA) or a fragment thereof which is capable of acting as a template for the isothermal amplification assay (e.g., LAMP assay) in the method of the disclosure. When used as a template in the isothermal amplification assay alongside sample templates, the positive control can be used to confirm that the isothermal amplification assay or assays have worked. In accordance with an example in which a LAMP assay is used, the isothermal amplification assay positive control may be purified genomic DNA obtained from Tilletia indica. In another example, the isothermal amplification assay positive control may be an isolated fragment of Tilletia indica DNA to which the primers in the reagent mixture can bind during the isothermal amplification assay. In one example, the isothermal amplification assay positive control is synthesized (i.e., a synthetic nucleic acid). In one example, the isothermal amplification assay positive control is produced by PCR in accordance with the Examples herein. In one example, the isothermal amplification assay positive control comprises the sequence set forth in SEQ ID NO: 13.

### Lateral Flow Test Strips and Test Apparatus

A test kit according to one or more embodiments of the present disclosure comprises one or more lateral flow test strips. Each lateral flow test strip may be formed of any material which permits flow of a liquid sample through by capillary action and which is known to be suitable for use in lateral flow devices. Such materials have been widely used in commercially-available diagnostic tests and will be known to a person skilled in the art. One such exemplary material may be a nitrocellulose membrane.

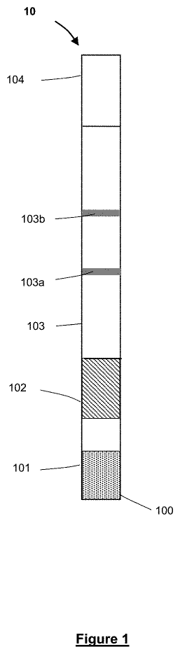

Each lateral flow test strip may comprise a label-holding portion, a test portion and a sample receiving portion. The lateral flow test strips may also comprise a control portion. An exemplary test strip is illustrated in FIG. 1. The size of the label-holding portion, the test portion, the sample receiving portion and the control portion may be adapted as necessary. For example, the precise dimensions of each may be adapted according to the particular dimensions of the lateral flow test strip(s) used and/or the dimensions of an apparatus with which the lateral test strip is used (if any).

The label-holding portion and the test portion are configured on the lateral flow test strips such that a sample to be tested contacts the label-holding portion before the test portion. Further, the sample-receiving portion is configured on the lateral flow test strips such that sample contacts the sample-receiving portion before the label-holding portion. The sample may also contact the control portion after contacting the test portion. Alternative configurations are possible, including configurations where multiple lateral flow test strips are used together.

As used herein, the terms “downstream” and “upstream”, when referring to the location of the various portions of the lateral flow test strip, will be understood to mean relative to the direction of flow of the sample through or along the lateral flow test strip.

Each lateral flow test strip according to one or more embodiments of the present disclosure may also comprise a fluid sink, which may act to draw the sample through or along the test strip(s).

As described herein, the lateral flow test strip(s) may be configured to include one or more capture reagents. Capture reagents used in accordance with one or more embodiments of the present disclosure may be any one of more agents having the capacity to bind an analyte of interest in a sample and thereby form a binding complex. Some examples of binding complexes include, but are not limited to, an antibody and hapten; antibody and antigen (wherein the antigen may be, for example, a peptide sequence or a protein sequence); complementary nucleotide or peptide sequences; polymeric acids and bases; dyes and protein binders; peptides and protein binders; enzymes and cofactors, and ligand and receptor molecules, wherein the term receptor refers to any compound or composition capable of recognising a particular molecule configuration, such as an epitopic or determinant site.

The label-holding portion of each lateral flow test strip comprises a mobilisable capture reagent configured to bind specifically to a hapten e.g., a fluorophore as described herein or non-fluorescent molecule as described herein. Accordingly, the mobilisable capture reagent will be configured to bind to the hapten-labelled nucleic acid probe of the test kit (which may be complexed with an amplification product, such as a LAMP product) to form a labelled binding complex. In one example, the hapten is FITC and the mobilisable capture reagent is an anti-FITC antibody. In another example, the hapten is digoxigenin and the mobilisable capture reagent is an anti-digoxigenin antibody. In another example, the hapten is carboxyfluorescein (FAM) and the mobilisable capture reagent is an anti-FAM antibody. However, other haptens for which commercially-available antibodies are available are described and contemplated herein. The term “mobilisable” shall be understood to mean that the capture reagent is capable of moving with the sample from the label-holding portion to the test portion and/or the control portion. The mobilisable capture reagent may be deposited at the label-holding portion prior to use of the lateral flow test strip by any suitable means known in the art.

The lateral flow test strip may also comprise an immobilised capture reagent at the test portion which is configured to bind specifically to biotin (e.g., present within the amplification product, such as LAMP product, in a sample). In this way, the test portion is configured to capture binding complexes comprising the amplification product e.g., complexes comprising the amplification product and the mobilisable capture reagent. Any suitable biotin ligand known in the art may be used, such as for example, avidin, streptavidin or derivatives thereof. The term “immobilised”, as used with respect to a capture reagent, will be understood to mean that the reagent is attached to the lateral flow test strip such that lateral flow of fluids through or along the test strip during an assay process will not dislodge the reagent. The capture reagent may be immobilised by any suitable means known in the art.

As used herein, the term “specifically binds”, “binds specifically”, “binds to specifically” or similar may refer to a capture reagent that does not bind significantly (e.g., above background binding levels) to any sample components other than the desired component or analyte. Accordingly, by way of example, a capture reagent which is “configured to specifically bind biotin” will not bind significantly or at all to any other analytes or components in a sample other than biotin.

The skilled person will be aware that an “antibody” is generally considered to be a protein that comprises a variable region made up of a plurality of immunoglobulin chains, e.g., a polypeptide comprising a V_{L} and a polypeptide comprising a V_{H}. An antibody also generally comprises constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. As used herein, the term “antibody” is also intended to include formats other than full-length, intact or whole antibody molecules, such as Fab, F(ab′)2, and Fv which are capable of binding the epitopic determinant. These formats may be referred to as antibody “fragments”. In accordance with one or more embodiments of the present disclosure, it will be expected that antibody fragments retain some or all of the ability of the corresponding full-length, intact or whole antibody to selectively bind to the hapten conjugated to the nucleic acid probe e.g., FITC, FAM or digoxigenin, as required, examples of which include, but are not limited to, the following:

(1) Fab, the fragment which contains a monovalent binding fragment of an antibody molecule and which can be produced by digestion of whole antibody with the enzyme papain to yield an intact light chain and a portion of one heavy chain;

(2) Fab′, the fragment of an antibody molecule which can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains;

(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO 94/07921 and WO 98/44001); and

(6) Single domain antibody, typically a variable heavy domain devoid of a light chain.

Accordingly, an antibody used as a capture reagent in accordance with one or more embodiments of the present disclosure may include separate heavy chains, light chains, Fab, Fab′, F(ab′)2, Fc, a variable light domain devoid of any heavy chain, a variable heavy domain devoid of a light chain and Fv. Such fragments can be produced by recombinant DNA techniques, or by enzymatic or chemical separation of intact immunoglobulins.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including a Fc region. The constant domains may be wild-type sequence constant domains or amino acid sequence variants thereof.

As described herein, the label holding portion of the lateral flow test strip comprises a mobilisable capture reagent configured to bind specifically to a hapten e.g., a fluorophore or digoxigenin. The mobilisable capture reagent may be conjugated to a detectable label to facilitate detection of an amplification product (e.g., LAMP product), when part of a binding complex comprising a hapten-labelled probe, during use of the test strip or an apparatus comprising same.

Suitable detectable labels for use in diagnostic applications are known in the art. Suitable detectable labels for use in accordance with one or more embodiments of the present disclosure may include, for example, particulate labels, radiolabels, fluorescent labels, enzymatic labels and imaging agents. For example, the labels may comprise latex or gold. For example, the labels may be latex beads (of any colour, including of two or more distinguishable colours). The labels may also be nanoparticles. For example, the labels may be gold nanoparticles. Any suitable nanoparticle may be used. The labels may be fluorescent labels e.g., a fluorescent molecule. Where the lateral flow test strip incorporates multiple fluorescent molecules, the respective molecules may be selected to fluoresce at different wavelengths e.g., upon excitation by light, to enable differential detection of two or more analytes in the sample. The labels may be reflective. Where the lateral flow test strip incorporates multiple reflective molecules, the respective molecules may be selected to reflect light at different wavelengths to enable differential detection of two or more analytes in the sample.

The mobilisable capture reagent of the label-holding portion may be conjugated to detectable labels by any means known in the art. For example, the detectable label may be conjugated to the mobilisable capture reagent via a suitable linker.

In some embodiments, the lateral flow test strips of the disclosure may be configured for use with an apparatus. The apparatus may be a device that operates as a single unit. For example, the apparatus may be provided in the form of a hand-held device. The apparatus may be a single-use, disposable, device. Alternatively, the apparatus may be partly or entirely re-usable. While in some embodiments the apparatus may be implemented in a laboratory, the apparatus may designed as a ‘end-use’ device, for home use or use in a workplace, etc. The apparatus may provide a rapid-test device, with identification of target conditions being provided to the user relatively quickly, e.g., in under 10 minutes, 5 minutes or under 1 minute

The apparatus may comprise a single test strip, or multiple test strips. For example, an apparatus comprising multiple test strips may comprise 2, 3, 4, 5, 6, 7, 8, 9, 10, 15, 20 or more test strips. The test strips may be arranged in parallel or in a series. The apparatus may also be configured such as that test strips can be replaced after use.

A lateral flow test strip according to one embodiment of the present disclosure in which the isothermal amplification assay is a LAMP assay is illustrated in FIG. 1 (test strip 10). The test strip 10 is a lateral flow test strip constructed of chemically-treated nitrocellulose, located on a waterproof substrate which is configured to detect the presence or absence of a LAMP product when complexed with a FITC-labelled nucleic acid probe in a liquid sample. The liquid sample will comprise the contents of the LAMP reaction mixture following performance of the LAMP assay and subsequent incubation with the nucleic acid probe.

Referring to FIG. 1, the test strip 10 is a lateral flow test strip including different zones arranged sequentially along the length of the strip, including a sample receiving zone 101 at the sampling end 100, a label-holding zone 102, a test zone 103, and a sink 104. The zones 101-104 comprise chemically-treated nitrocellulose, located on a waterproof substrate 105. The arrangement of the test zones 101-104 and substrate 105 is such that, when contacted with the sample receiving zone 101, the liquid sample is absorbed into the sampling receiving zone 101 and at least part of the sample travels under capillary action sequentially through the sample receiving zone 101, the label-holding zone 102, and the test zone 103 and accumulates finally at the sink 104.

In this embodiment, the label-holding zone 102 comprises a label-conjugated antibody (i.e., the mobilisable capture reagent). The label-conjugated antibody is an anti-FITC antibody designed to bind specifically to the FITC label of the nucleic acid probe in the sample. Accordingly, as the liquid sample travels through the label-holding zone 102, the FITC-labelled nucleic acid probe present therein (including FITC-labelled nucleic acid probe complexed with the LAMP product) binds to the label-conjugated anti-FITC antibody to form a labelled binding complex. When the labelled binding complex comprises the FITC-labelled nucleic acid probe complexed with the LAMP product, the labelled binding complex shall be referred to as the “labelled LAMP complex”. When the labelled binding complex comprises the FITC-labelled nucleic acid probe only, the labelled binding complex shall be referred to as the “labelled probe complex”. Collectively, these may be referred to as the “labelled complexes”. The sample containing the labelled LAMP complex and/or labelled probe complex continues to travel though the test strip to the test zone 103 and contact the test stripe 103a (or other type of test portion) that contains immobilized compounds capable of binding biotin with high specificity and affinity i.e., avidin or streptavidin. On contact, the immobilized compounds in the first test stripe 103a binds to the biotin in the labelled LAMP complex to form a labelled LAMP sandwich. The liquid sample will continue through the test zone to contact a control stripe 103b which contains an immobilized reagent capable of binding the anti-FITC antibody. For example, if the anti-FITC antibody used to bind the FITC-labelled nucleic acid probe is a mouse anti-FITC antibody, the immobilized reagent at the control stripe 103b will be an anti-mouse antibody. Similarly, if the anti-FITC antibody used to bind the FITC-labelled nucleic acid probe is a rabbit anti-FITC antibody, the immobilized reagent at the control stripe 103b will be an anti-rabbit antibody, and so on. Accordingly, the immobilized reagent at the control stripe 103b may be configured to the choice of immobilisable capture reagent used to bind the FITC-labelled nucleic acid probe.

In this embodiment, the label-conjugated antibody is labelled with gold nanoparticles. As such, immobilisation of the label-conjugated antibody at the test stripe 103a and/or the control stripe 103b may be detected by the naked eye. Of course, in alternative embodiments, other types of labels may be used in place of gold nanoparticles, such as latex beads or quantum dots etc.

In the present embodiment, detectable signal at the test stripe 103a is indicative of the presence of labelled LAMP complex in the sample and an indication that a sample being tested is positive for Karnal bunt. Detectable signal at the control stripe 103b is indicative that an adequate amount of the sample that has flowed through the test strip and reached the control stripe 103b.

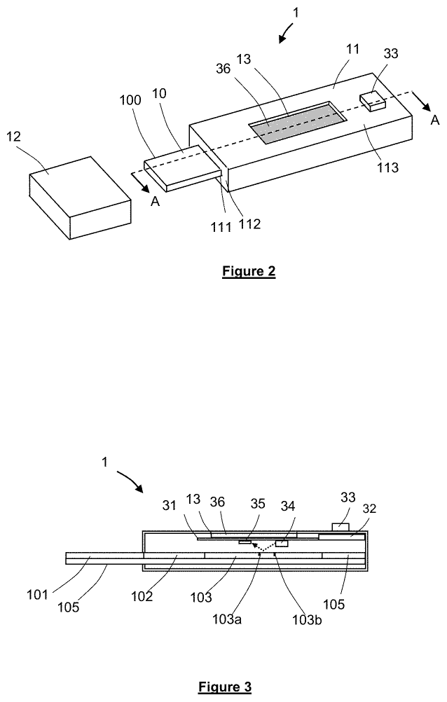

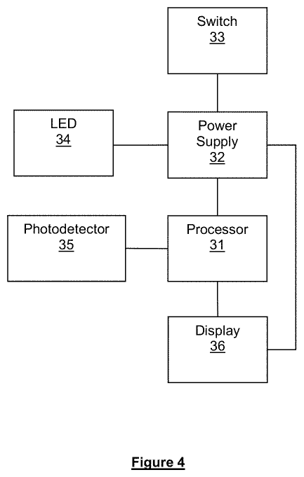

In an alternative embodiment, a test strip 10 of the disclosure may be used in combination with a device e.g., a handheld device, to assist in detection of Karnal bunt in a sample. An apparatus according to an embodiment of the present disclosure is illustrated in FIGS. 2 and 3 (test device 1). The test device 1 is a hand-held device configured for use with a test strip 10 as illustrated in FIG. 1 to detect the presence or absence of a LAMP product complexed with a FITC-labelled nucleic acid probe in a sample following performance of a LAMP assay.

The test device 1 includes an elongate lateral flow test strip 10 and a casing 11. The test strip 10 is partially housed in the casing 11 with a sampling end 100 of the test strip 10 protruding from an opening 111 in an end surface 112 of the casing 11, allowing sample to be received directly thereon. The sampling end 100 of the test strip 10 is coverable by a cap 12. The test device 1 also includes an LCD display 36 visible through an opening 13 in a top surface 113 of the casing 11 for displaying results of testing.

Referring to FIGS. 1 and 3, the test strip 10 is a lateral flow test strip including different zones arranged sequentially along the length of the strip, including a sample receiving zone 101 at the sampling end 100, a label-holding zone 102, a test zone 103, and a sink 104. The zones 101-104 comprise chemically-treated nitrocellulose, located on a waterproof substrate 105. The arrangement of the test zones 101-104 and substrate 105 is such that, when contacted with the sample receiving zone 101, the liquid sample is absorbed into the sampling receiving zone 101 and at least part of the sample travels under capillary action sequentially through the sample receiving zone 101, the label-holding zone 102, and the test zone 103 and accumulates finally at the sink 104.

The label-holding zone 102 comprises a label-conjugated antibody (i.e., the mobilisable capture reagent). The label-conjugated antibody is an anti-FITC antibody designed to bind specifically to the FITC label of the nucleic acid probe in the sample. Accordingly, as the liquid sample travels through the label-holding zone 102, the FITC-labelled nucleic acid probe present therein (including FITC-labelled nucleic acid probe complexed with the LAMP product) binds to the label-conjugated anti-FITC antibody to form a labelled binding complex. When the labelled binding complex comprises the FITC-labelled nucleic acid probe complexed with the LAMP product, the labelled binding complex shall be referred to as the “labelled LAMP complex”. When the labelled binding complex comprises the FITC-labelled nucleic acid probe only, the labelled binding complex shall be referred to as the “labelled probe complex”. Collectively, these may be referred to as the “labelled complexes”. The sample containing the labelled LAMP complex and/or labelled probe complex continues to travel though the test strip to the test zone 103 and contact the test stripe 103a (or other type of test portion) that contains immobilized compounds capable of binding biotin with high specificity and affinity i.e., avidin or streptavidin. On contact, the immobilized compounds in the first test stripe 103a binds to the biotin in the labelled LAMP complex to form a labelled LAMP sandwich. The liquid sample will continue through the test zone to contact a control stripe 103b which contains an immobilized reagent capable of binding the anti-FITC antibody. For example, if the anti-FITC antibody used to bind the FITC-labelled nucleic acid probe is a mouse anti-FITC antibody, the immobilized reagent at the control stripe 103b will be an anti-mouse antibody. Similarly, if the anti-FITC antibody used to bind the FITC-labelled nucleic acid probe is a rabbit anti-FITC antibody, the immobilized reagent at the control stripe 103b will be an anti-rabbit antibody, and so on. Accordingly, the immobilized reagent at the control stripe 103b may be configured to the choice of immobilisable capture reagent used to bind the FITC-labelled nucleic acid probe.

In this particular embodiment, the label-conjugated antibody is labelled with a fluorescent quantum dot (QD) that fluoresces at a specific emission peak wavelength following UV light excitation (e.g., at 525, 625 or 800 nm). Of course, in alternative embodiments, other types of labels may be used in place of quantum dots, such as latex beads or gold particles, etc., and/or other specific emission peak wavelengths may be used.

By illuminating the stripes 103a, 103b with UV light, the presence of the QD label will result in a detectable light emission at a known emission peak wavelength. The intensity of the light emission (the size of the peaks) is indicative of the number of labelled complexes/antibodies bound to the stripes. Detectable light emission at the test stripe 103a is indicative of the presence of labelled LAMP complex in the sample and an indication that a sample being tested is positive for Karnal bunt. Detectable light emission at the control stripe 103b is indicative that an adequate amount of the sample that has flowed through the test strip and reached the control stripe 103b. As such, one or more wavelength sensitive photodetectors, forming part of a reader, can be used in the test device 1 to identify the presence amount of labelled LAMP complex in the sample through monitoring of the test stripe 103a. The one or more photodetectors can also be used to determine, through monitoring of the control stripe 103b, that a sufficient amount of sample has travelled through the test stripe 103a to the control stripe 103b and that binding of the labelled complexes has been successful.

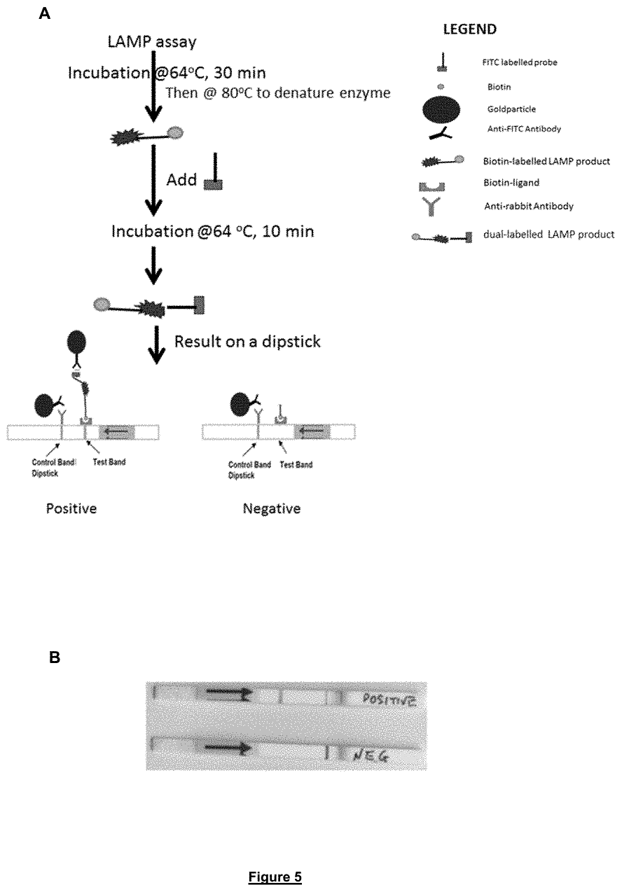

Referring to FIGS. 3 and 4, a reading apparatus of test device 1 of the present embodiment is now described in more detail. The reading apparatus includes a printed circuit board having a processor 31, a power supply (battery) 32, a switch 33, a UV LED 34, a multi-wavelength photodetector 35 and the display 36. The LED 34 is configured to emit light in the UV spectrum (at about 300 to 400 nm) that is incident on the stripes 103a and 103b to cause excitation of any quantum dot labels located thereon. The multi-wavelength photodetector 35 in combination with the processor 31 is configured to detect the different intensities of light emitted from the quantum dots at different distinct wavelengths (if desired).

In use, the cap 12 is removed from sampling end 100 of the test strip and a liquid sample is directed onto the sample receiving zone 101. The cap 12 can be replaced and, after approximately 1 or 2 minutes, giving sufficient time for the lateral flow process to take place, the switch 33 can be depressed, causing flow of electricity from the power supply 32 to the LED 34, resulting in emission of UV light from the LED 34 that is incident on the stripes 103a, 103b of the test strip 10. The UV light results in excitation of any or all of the quantum dots that may be immobilized as part of the labelled complexes at the stripes 103a, 103b causing light emission at respective wavelength peaks. In combination with the multi-wavelength photodetector 35, the processor 31 is configured to determine the size of the emission peaks and identify from this (a) if the sample mix has arrived at the control stripe 103c and labelling has been effective, and if yes, identify (b) the presence and optionally, an amount, of labelled LAMP complex present in the sample based on the intensity of light emission detected at test stripe 103a.

While a manual switch 33 is described above, in alternative embodiments, switching may be automated. For example, switching may be configured to occur upon replacement of the cap 12 onto the casing 11 or due to fluid activation, as the sample travels through a fluid-activated switch that may be provided in the device.

The LED may be carefully calibrated to ensure that the light emission from the LED is consistent from one device to the next, ensuring that a degree of excitation of the quantum dots is consistent. Additionally, or alternatively, a calibration mechanism may be integrated into the device. A known quantity of quantum dots, configured to fluoresce at yet another wavelength, may be immobilized on the test strip, e.g. at a further test stripe. Depending on the intensity of the fluorescence detected from the known quantity of quantum dots, the processor may adjust its interpretation of the light emission from quantum dots on the labelled complexes. Additionally, or alternatively, multiple LEDs may be used to excite the quantum dots with a view to suppressing the overall effect of any rogue LEDs.

If, during use, it is identified there is insufficient amount of sample to reach the control stripe, the processor 31 is configured to cause the display 36 to present the words INVALID TEST. In this respect, the processor 31, in combination with the multi-wavelength photodetector 35, is configured to determine the size of the emission peaks at the control stripe 103c and identify from this (i) if the sample has arrived at the control stripe 103c, and/or (ii) if labelling has been effective.

If, during use, it is identified there is sufficient amount of sample and labelling is effective, the processor 31 is configured to provide a determination that the sample is contaminated with karnal bunt or not.

Since the device of the present embodiment is a hand-held device, the device may be used in the laboratory, at home or in the workplace.

The device is configured to allow removal of a used test strip from the casing 10, via the opening 111, and allow placement of a new test strip into the casing 10, via the same opening 111. In alternative embodiments, the device may be entirely a single-use device.

Embodiments described and illustrated herein refer to the use of a lateral flow test strip in a method of the disclosure employing a LAMP assay. However, it will be understood that the lateral flow test strip of the disclosure is suitable for use with any isothermal amplification assay format as described herein e.g., such as RPA and HBA.

Diagnostic Methods

The test kit of the disclosure may be used in a diagnostic method to determine whether or not a sample is contaminated with Karnal bunt or spores thereof. The method may be carried out in a laboratory setting, home, workplace or other environment.

The diagnostic method of the disclosure broadly comprises the following steps:
(a) performing an isothermal amplification assay on a nucleic acid e.g., DNA, extracted from a sample (sample DNA) using the reagent mixture of the test kit described herein;
(b) incubating the product of (a) in the presence of the nucleic acid probe of the test kit described herein;
(c) contacting the product of (b) with the sample receiving portion of a lateral flow test strip of the test kit described herein;
(d) detecting the presence or absence of an amplification product at the test portion of the lateral flow test strip; and
(e) determining the presence or absence of Tilletia indica nucleic acids in the sample based on the presence or absence of an amplification product at the test portion.

As described herein, any isothermal amplification assay known in the art may be used. A number of exemplary isothermal amplification assays are described herein in the context of a test kit of the disclosure and shall be taken to apply mutatis mutandis to each and every example describing a method of the disclosure. In one example, the isothermal amplification assay is selected from a LAMP assay, a HDA assay and a RPA assay. In one example, the isothermal amplification assay is a LAMP assay.

In accordance with an example in which the reagent mixture of the test kit is provided in dried form, water is added to the reagent mixture prior to adding the sample DNA and incubating the reagent mixture to perform the isothermal amplification assay. The final concentration of each component in an exemplary reagent mixture of the test kit is described in Tables 2 and 3 herein.

As will be appreciated by a person skilled in the art, the incubation during an isothermal amplification assay (e.g., a LAMP assay) is isothermal. In one example, the isothermal amplification assay at step (a) of the method is performed at about 60° C. to about 66° C. For example, the isothermal amplification assay at step (a) of the method may be performed at 60° C. For example, the isothermal amplification assay at step (a) of the method may be performed at 61° C. For example, the isothermal amplification assay at step (a) of the method may be performed at 62° C. For example, the isothermal amplification assay at step (a) of the method may be performed at 63° C. For example, the isothermal amplification assay at step (a) of the method may be performed at 64° C. For example, the isothermal amplification assay at step (a) of the method may be performed at 65° C. For example, the isothermal amplification assay at step (a) of the method may be performed at 66° C.

The duration of incubation of the isothermal amplification assay may be performed for any amount of time sufficient to produce an amplification product in the presence of appropriate template DNA from Tilletia indica. In one example, the duration of incubation is at least about 15 minutes. In another example, the duration of incubation is at least about 20 minutes. Preferably, the duration of incubation is at least about 30 minutes. In each case, the incubation step will be for an amount of time sufficient to produce enough amplification product for detection in the downstream steps using the lateral flow test strip.

Once the isothermal amplification assay at step (a) has been performed, the nucleic acid probe of the test kit is added to the amplification assay mixture. Since the nucleic acid probe is provided in the test kit in a dried form, before use the nucleic acid probe is resuspended in an appropriate diluent e.g., TE, Tris or water, to achieve a desired working concentration e.g., 5 μM. In one example, about 5 picomoles to about 40 picomoles of the nucleic acid probe is added to the isothermal amplification assay mixture. In a preferred example, about 20 picomoles of the nucleic acid probe is added to the isothermal amplification assay mixture.

The isothermal amplification reaction mixture is then allowed to incubate in the presence of the nucleic acid probe for a further period sufficient for the nucleic acid probe to hybridise to the amplification product i.e., step (b). For example, the incubation at step (b) may proceed for about 5 minutes, about 10 minutes, about 15 minutes, about 20 minutes, about 30 minutes or longer as required in order to achieve hybridisation of the nucleic acid probe to the amplification product (if present).

The incubation at step (b) may conveniently occur at the same or approximately the same temperature that the isothermal amplification assay was performed in order to achieve hybridisation of the nucleic acid probe to the amplification product (if present). In accordance with one example in which a LAMP assay is performed with an incubation at step (a) which is at approximately 64° C., the incubation at step (b) is also performed at approximately 64° C.

Following completion of step (b), the method comprises aliquoting a small volume of the isothermal amplification reaction mixture from step (b) into a container or tube containing a volume of running buffer, or alternatively adding a volume of running buffer to the isothermal amplification reaction mixture, to produce a spiked running buffer. The volume of isothermal amplification reaction mixture combined with the running buffer may vary provided that there is sufficient reaction mixture (and if present, amplification product) to be detected by the lateral flow test strip. However, in one example, the volume of isothermal amplification reaction combined with the running buffer is at least about 2 μl. In some examples, 20 μl or more of the LAMP reaction may be added to the running buffer. Once the isothermal amplification reaction mixture and running buffer are combined and mixed, the sample receiving portion of a lateral flow test strip from the test kit is then dipped into the spiked running buffer. The lateral flow test strip is then allowed to incubate at ambient temperature for a period of time sufficient for the spiked running buffer to travel up the length of the lateral flow test strip and reach the control portion.

The presence of a signal at the test portion of the lateral flow test strip owing to the binding of the amplification product thereto is indicative of the presence of Tilletia indica nucleic acids in the sample. Likewise, an absence of signal at the test portion of the lateral flow test strip is indicative of the absence of Tilletia indica nucleic acids in the sample. In this way, a determination regarding the presence or absence of Tilletia indica nucleic acids in the sample can be made based on the presence or absence of an amplification product at the test portion of the lateral flow test strip.

Furthermore, the detection of a signal at the control portion of the test strip is indicative that the sample has flowed through the lateral flow test strip as desired.

The diagnostic method of the disclosure may further comprise the step of isolating or extracting DNA from the sample prior to performing step (a). Any method known to be suitable for extracting DNA from fungal spores may be used. In one example, the ‘freeze-thaw’ method described in Example 3 may be employed. In another example, a commercially-available DNA extraction kit may be employed.

The diagnostic method of the disclosure may be used to detect the presence of contaminating Tilletia indica and/or spores thereof in a range of samples. In one example, the sample to be tested is grain e.g., wheat or triticale, or a part thereof. The wheat may be any species or variety of wheat, including but not limited to those selected from the group consisting of Triticum aestivum, Triticum aethiopicum, Triticum araraticum, Triticum boeoticum, Triticum carthlicum, Triticum compactum, Triticum dicoccoides, Triticum dicoccon, Triticum durum, Triticum ispahanicum, Triticum karamyschevii, Triticum macha, Triticum militinae, Triticum monococcum, Triticum polonicum, Triticum spelta, Triticum sphaerococcum, Triticum timopheevii, Triticum turanicum, Triticum turgidum, Triticum Urartu, Triticum vavilovii, and Triticum zhukovskyi. The triticale may be any species triticale, including but not limited to those selected from the group consisting of Triticosecale blaringhemii, Triticosecale neoblaringhemii, Triticosecale schlanstedtense, and Triticosecale semisecale. In another example, the sample to be tested is a commodity comprising grain or a part thereof e.g., a foodstuff or feedstock. In another example, the sample to be tested is debris suspected of comprising grain or a part thereof as a contaminant e.g., from a shipping container or agricultural equipment. In yet other examples, grain or a part thereof is washed with a solution (e.g., an aqueous solution) and the sample to be tested is debris collected from the solution post wash (e.g., using a filter or sieve). In accordance with this example, the debris collected from the solution may comprise one or more spores from Tilletia indica. However, it will be appreciated that the method of the disclosure may be used to detect the presence or absence of contaminating Tilletia indica and/or spores thereof in any sample suspected of being contaminated.

### EXAMPLES

### Example 1—Development of a Lateral Flow Test Kit and Assay for Detection of Karnal Bunt (T. indica)

This example describes a test kit and protocol for detection of Karnal bunt in a sample based on LAMP assay and a lateral flow test strip. Specifically, the present inventors have developed a test kit and diagnostic protocol which is based on the LAMP assay previously described in Tan et al., (2016) PLoS ONE 11(11):e0166086, albeit modified so that a determination regarding the presence or absence of karnal bunt in a sample can be made in the absence of expensive equipment using lateral flow test strip technology. The test kit has also been designed so that it may be assembled as a freezer kit or a lyophilised it that can be transported at ambient temperatures.

The test kit has been designed to comprise the following components:
1. LAMP assay reagent mixture (Master Mix) (lyophilised or liquid);
2. Hybridisation probe (dried or lyophilised);
3. Lateral flow test strip;
4. Lateral flow test strip buffer; and
5. A positive control DNA (dried or lyophilised) (optional component).

### LAMP Assay Reagent Mixture

The test kit and LAMP protocol relies on a LAMP assay reagent mixture comprising six primers previously described in Tan et al., (2016) PLoS ONE 11(11):e0166086, with the exception that one of the primers has been modified in order to amplify a biotinylated LAMP product. Accordingly, the inventors prepared a LAMP assay reagent mixture comprising the primer set forth in Table 1 below.

TABLE 1
LAMP assay primers

| Primer | | |
|---|---|---|
| ID | Sequence (5′-3′) | SEQ ID NO: |
| Ti-FIP | [Btn]GAATAGTACTTGATATTTTACTTT | SEQ OD NO: 2 |
| | TGGGGCTCATTGATTCTACATATTATCTT | |
| | TAC | |
| Ti-BIP | GCTATTCAGATTATAAATCGAAGATAAAG | SEQ OD NO: 3 |
| | ACCTTCCTTTATTTTGGAACTA | |
| Ti-LF | GATATCTAAACACATTTGAATTAGAAAG | SEQ OD NO: 4 |
| Ti-LB | AATAGCAAAGGTGTAATAACGA | SEQ OD NO: 5 |
| Ti-F3 | CCAAACTACGATATAGCAATATATC | SEQ OD NO: 6 |
| Ti-B3 | TTATAGTTACTAAGTGTATTTGATGTTC | SEQ OD NO: 7 |

The complete LAMP assay reaction mixture developed by the inventors is described in more detail in Table 2 below.

TABLE 2
Composition of LAMP reagent mixture for one reaction

| 1XAssay Mix | Final conc | Vol/rxn |
|---|---|---|
| *LAMP Buffer | 1X | 2.5 |
| MgSO4 | 6 mM | 1.5 |
| dNTPs | 1.4 mM | 3.5 |
| Ti-FIP-Biotin labelled primer | 1.6 μM | 0.4 |
| Ti-BIP primer | 1.6 μM | 0.4 |
| Ti-LF primer | 0.4 μM | 1 |
| Ti-LB primer | 0.4 μM | 1 |
| Ti-F3 primer | 0.2 μM | 0.5 |
| Ti-B3 primer | 0.2 μM | 0.5 |
| Bst 2.0 WarmStart ® DNA Polymerase | 8U/RXN | 1 |
| Large Fragment (8000 U/ml) | | |
| milliQ Water | | 7.7 |
| TOTAL | | 20 |

*The LAMP buffer used was a 1X LAMP buffer provided with the Bst 2.0 WarmStart ® DNA Polymerase Large Fragment. This buffer comprised 20 mM Tris-HCl, 10 mM (NH₄)₂SO₄, 50 mM KCl, 2 mM MgSO₄, 0.1% Tween ® 20, (pH 8.8 at 25° C.). The LAMP buffer may differ depending on the DNA polymerase used e.g., may be any appropriate isothermal buffer provided by the manufacturer with a DNA polymerase.

When provided in a lyophilised form, the LAMP assay reaction mixture may be prepared in accordance with the protocol described in Example 2 and Table 3 below. Prior to use, a lyophilised LAMP reaction mixture is resuspended in an appropriate volume of sterile MilliQ H2O, taking into consideration the volume of template DNA to give a total reaction volume of 25 μL per reaction.

### Hybridisation Probe

The inventors also designed and tested a unique hybridisation probe specific for Tilletia indica capable of binding the LAMP product produced by the primers in Table 1 with good specificity, at the same reaction temperature used in the LAMP assay. The hybridisation probe was designed to target a unique AT rich (75.11% AT rich) region in the mitochondrial genome of Tilletia indica. More specifically, the unique hybridisation probe was designed based on the following criteria:
it must not hybridise to any of the six primers in the LAMP assay;
it must have sufficient GC content for specific hybridisation at the same temperature as the LAMP assay (i.e., at 64° C.);
it must not form a secondary structure strong or significant enough to hamper hybridization to the target region; and
it must not result in the formation of primer dimer.

Based on these criteria, the inventors designed the following hybridisation probe: GTGTAATAACGACTAGTTCCA (SEQ ID NO: 8), and labelled it with a 5′ fluorescein isothiocyanate (FITC). This probe was found to have a very weak secondary structure and did not result in the formation of primer dimer. The probe's GC content is 38.10%, which is low due to the nature of the target region, and has a Tm of 53.4° C. Based on these parameters the inventors predicted that the probe would bind to the LAMP product with a very high specificity since the hybridization temperature of 64° C. is more than 10° C. higher than the Tm of the probe, thereby eliminating any possibility of a false positive due to non-specific binding. The hybridisation probe set forth in SEQ ID NO: 8 was chosen for inclusion in the test kit. However, the inventors also designed two further hybridisation probes which met the design criteria and which may be considered for inclusion in a test kit of the disclosure. These alternative hybridisation probes comprise the sequences set forth in SEQ ID NOs 9 and 10 respectively. These probe sequences may be labelled with a 5′ FITC and included in the test kit as additional or alternative probes.

Stable, dried hybridisation probe was prepared for inclusion in a test kit. Tubes containing sufficient hybridisation probe for eight reactions was prepared. To each tube 8 μl of hybridisation probe (20 μM) was combined with 1.5 μl 1% PVA (CatP8136, Sigma-Aldrich), 200 mM TrisHCl, pH 8.0. The tubes were then placed in a tube rack, covered with aluminium foil and dried on a clean bench area overnight at room temperature. The resulting dried hybridisation probe is stable up to 4 years (Ivanova and Kuzmina (2013) Protocols for dry DNA storage and shipment at room temperature. Molecular Ecology Resources (2013) doi: 10.1111/1755-0998.12134).

Before use, the dried hybridisation probe in each tube is resuspended in 32 μl TE or water and 4 μl is used in each LAMP assay.

### Positive Control DNA

A stable freeze-dried positive control DNA was also prepared for use in the kit and diagnostic protocol (as an optional feature).

The positive control DNA is a piece of mitochondrial DNA that is present in all isolates of T. indica tested to date. The positive control DNA was prepared by PCR. Each PCR reaction was performed in a 10 μL volume containing 2 mM MgCl₂, 0.2 mM of each of the four dNTPs (dATP, dTTP, dCTP and dTTP), 5 pmol of forward primer: CTAATTCTTTTACCTGAGGTGC (SEQ ID NO: 11); 5 pmol of reverse primer: AGTTACTAAGTGTATTTGATGTTC (SEQ ID NO: 12), ˜10 ng of genomic T. indica DNA and 1 unit of Taq DNA polymerase (Thermo Fisher, Scientific) in a buffer (50 mM Tris, pH 9.0, 20 mM NaCl, 1% Trition X-100, 0.1% gelatine). The thermocycle profile was as follows: an initial denaturation cycle of 94° C. for 3 min; 35 cycles of 94° C. for 30 s (denaturation), annealing temperature at 50° C. for 30 s, 72° C. for 45 s (extension); and a final extension step of 72° C. for 10 min.

The expected amplicon was 370 bp. This was confirmed by electrophoresis on a 1% agarose gel, after which the PCR amplicon was purified using QIAquick PCR Purification Kit (Qiagen). The purified PCR amplicon was quantitated using a Qubit fluorometer and the concentration diluted to 0.1 ng/μl.

To prepare dried positive control DNA for inclusion in a test kit, 3 μl of the purified PCR amplicon (0.1 ng/μ1) was combined with 1.5 μl 1% PVA (CatP8136, Sigma-Aldrich), 200 mM TrisHCl, pH 8.0. This was then aliquoted into 3 microtubes, each containing 1.5 μl of the PCR amplicon mixture. The microtubes were then placed in a tube rack, covered with aluminium foil and dried on a clean bench area overnight at room temperature. The resulting dried control DNA is stable up to 4 years (Ivanova and Kuzmina (2013).

Before use, each control DNA in each tube is resuspended in 5 μl TE or water to be used as control template in a positive control reaction.

### Lateral Flow Test Strip and Buffer

As the test kit was designed for use with lateral flow test strip, the test kit further includes the Milenia HybriDetect universal test strip (dipstick) or similar, which is based on lateral flow technology with gold particles. These lateral flow test strips are designed to detect a biotin and FITC/FAM labelled analyte in a sample. Any lateral flow test strip having these qualities would be suitable.

The test kit also included a buffer (a running buffer) to assist the LAMP assay reaction mixture (containing the LAMP product analyte) travel through the lateral flow test strip. The running buffer included was the LFA buffer provided with the Milenia HybriDetect universal test strip. However, any suitable running buffer known in the art may be used e.g., PBS.

### Diagnostic Protocol

Having designed the Karnal bunt test kit and diagnostic protocol, the inventors then tested the ability of the test kit and protocol to accurately detect Tilletia indica DNA in a sample.

Template Tilletia indica DNA was first prepared from bunted grain. First, teliospores were extracted from seeds/grain using the size-selective sieve wash test as described in Section 3.2 of ISPM 27 Diagnostic Protocols DP 4: Tilletia indica Mitra (2014). 200 μl of TE pH 8.0 was then pipetted onto the pellet in the tube, after which time the tub was capped shut. However, the amount of TE (or MilliQ water) added to the pellet may vary depending on the size of the pellet. For example, for a less heavily bunted grain a volume of about 20 μl TE pH 8.0 may suffice. The tube was then immersed in an ice-salt bath for 5 minutes, and then immediately placed in a boiling water bath for a further 5 minutes (a heat block could also be used). This freeze-thaw cycles was repeated three times to release DNA from the fungal spores. 5 μl of the resulting solution was used as template for subsequent LAMP assays.

To perform the LAMP assay, 5 μl of the control DNA or template Tilletia indica DNA (i.e., DNA from a sample being tested) in 1× ThermoPol Buffer (New England Biolabs) was added to 20 μl of LAMP reagent mixture. The LAMP reaction was then allowed to proceed by incubating the LAMP reagent mixture (25 μl) at 64° C. for 30 minutes and then at 80° C. for 10 min to denature the DNA polymerase. Following the LAMP assay, 4 μl of the FITC-labelled hybridisation probe (5 μM) was added (approx 20 picomoles) and the reaction mixture was incubated at 64° C. for a further 10 minutes in order to produce a LAMP product dual-labelled with biotin and FITC (only where Tilletia indica DNA was present in the LAMP assay). A volume of 20 μl of the reaction mixture was then added to 100 μl lateral flow assay (LFA) buffer (Milenia® HybriDetect, MGHD1, Milenia Biotec, Germany—this could equally be Tris-saline, PBS or other suitable buffer) in a 1.5 ml microtube and mixed gently with a pipette. A lateral flow test strip (Milenia® HybriDetect, MGHD1, Milenia Biotec, Germany) was then dipped into the LFA buffer containing the reaction mixture and the results visualized within 5-15 minutes.

The protocol steps are schematically illustrated in FIG. 5A and the results of the protocol are shown in FIG. 5B. As can be seen from the lateral flow test strips in FIG. 5B, the test kit and protocol is clearly capable of detecting the presence (positive) and absence (negative) of a LAMP product, indicating the presence or absence Tilletia indica DNA respectively in a sample.

The diagnostic test kit can be deployed in plant health diagnostic laboratories, grain processing centres, quarantine stations and farmers for routine screening of grain samples for exports, imported products and agricultural machineries that may potentially have contaminants with karnal bunt e.g. imported fertilizers, animal feeds, agricultural machineries (e.g. harvesters on lease from overseas). Screening for Karnal bunt is important to the health of the agriculture industry, particularly in countries which have significant wheat and triticale production, processing or consumer markets. The diagnostic test kit of the disclosure will improve high-throughput screening capabilities for Karnal bunt, such as may be required in an incursion scenario. The test kit of the disclosure may also help protect market access for Australia's wheat exports (refer to Pakistan incident in http://www.abc.net.au/pm/content/2004/s1053360.htm) and other countries which do not currently have karnal bunt. The availability of a simple kit may convince growers and the industry to have their wheat harvests routinely screened for exports. The kit will be valuable to protect market access for other overseas wheat growing countries with no Karnal bunt as well as to demonstrate areas of freedom to increase market access for those with a history of an incursion.

### Example 2—Development of a Lyophilized LAMP Reagent Mixture

In order to make the test kit more stable during storage and/or transport, the inventors also developed a version of the test kit in which the LAMP reagent mixture is provided in lyophilised form.

Parameters for the preparation of lyophilized assay mix were determined using a series of experiments. The effects of two candidate cryoprotective agents, sucrose and trehalose (Rey and May (2010) Eds, Freeze drying/Lyophilization of pharmaceutical and biological products, 3^{rd} Ed. Drugs and the Pharmaceutical Sciences Vol 206), on the performance of the LAMP reagent mixture during a LAMP assay were assessed at final concentrations of 2%, 4%, 6%, 8% and 10%. Both sucrose and trehalose were found to have no inhibitory effect on the LAMP assay when performed in accordance with Example 1.

The inventors then freeze dried a series of LAMP reagent mixture comprising sucrose or trehalose at concentration of 2.5%, 5%, 7.5% or 10% to determine the optimum percentage of cryoprotectant to be used for lyophilisation, as well as to compare the protective properties of the two agents. Freeze drying was performed using a FreeZone® 6 Litre Benchtop Freeze Dry Systems (Model 77520 Series) according to the manufacturer's instructions. Each serum vial containing lyophilised LAMP reagent mixture was then sealed under vacuum and stored at room temperature.

The lyophilised LAMP reagent mixtures were found to perform best in the diagnostic protocol when the respective cryoprotectants were present at final concentrations of 7.5%. That is, a more intense band was evident on the lateral flow test strips when returning a positive result, indicating that the LAMP assay was more efficient when the cryoprotectants were present in the LAMP reagent mixtures at 7.5%. Furthermore, LAMP assays performed with LAMP reagent mixtures containing trehalose at 7.5% appeared to result in a slightly more intense positive band compared to the corresponding LAMP assays performed with LAMP reagent mixtures containing sucrose at 7.5%. Based on these findings, a lyophilised LAMP reagent mixture containing 7.5% trehalose was determined as being optimum. However, sucrose at 7.5% was also deemed suitable.

Eight vials of the LAMP reagent mixture with final concentration of 7.5% trehalose (each vial containing sufficient mixture for eight reactions) were then prepared. Briefly, a mastermix was prepared according to the constituents and volumes set forth in Table 3. The mastermix was then vortexed thoroughly, spun down using a centrifuge, and aliquoted into eight 2 ml vials.

TABLE 3
Composition of Master Mix for 8 vials*

| | Conc per | | Total Vol(μl) in |
|---|---|---|---|
| Master Cocktail | 25 μL rxn | Vol(μl)/rxn | mastermix |
| **LAMP Buffer | 1X | 2.5 | 175 |
| MgSO₄ | 6 mM | 1.5 | 105 |
| dNTP | 1.4 mM | 3.5 | 245 |
| Ti-FIP-Biotin | 1.6 μM | 0.4 | 28 |
| Labelled | | | |
| Ti-BIP | 1.6 μM | 0.4 | 28 |
| Ti-LF | 0.4 μM | 0.1 | 7 |
| Ti-LB | 0.4 μM | 0.1 | 7 |
| Ti-F3 | 0.2 μM | 0.05 | 3.5 |
| Ti-B3 | 0.2 μM | 0.05 | 3.5 |
| Bst 2.0 | 8U/μM | 1.0 | 70 |
| WarmStart ® | | | |
| DNA Polymerase | | | |
| 25% Trehalose | 7.5% | 7.5 | 525 |
| (w/v) | | | |
| TOTAL | — | 17.1 | 1197 |

*Each vial contained sufficient mastermix for eight reactions, accounting for an additional 0.5 reactions per vial and a further 0.2 reactions to compensate for carry-over on pipette tips during pipetting.
**The LAMP buffer used was a 1X LAMP buffer provided with the Bst 2.0 WarmStart ® DNA Polymerase Large Fragment. This buffer comprised 20 mM Tris-HC1, 10 mM (NH₄)₂SO₄, 50 mM KC1, 2 mM MgSO₄, 0.1% Tween ® 20, (pH 8.8 at 25° C.). The LAMP buffer may differ depending on the DNA polymerase used e.g., may be any appropriate isothermal buffer provided by the manufacturer with a DNA polymerase.

Freeze drying was then performed using a FreeZone® 6 Litre Benchtop Freeze Dry Systems (Model 77520 Series) according to the manufacturer's instructions, after which time the vials were sealed under vacuum and stored at room temperature.

The vials containing lyophilised LAMP reagent mixture were then tested using the LAMP assay diagnostic protocol described in Example 1 on day 1 (quality testing), week 1, week 2, week 4, week 8, week 12 and week 16 (following lyophilisation), and compared to the results of the same protocol performed using fresh (non-lyophilised) LAMP reagent mixture. The fresh (non-lyophilised) LAMP reagent mixture was prepared according to Table 3, with the exception that 6.9 μl of sterile milliQ water was added per reaction. LAMP assays performed with fresh and lyophilised reagent mixtures were performed using 5 μl of Tilletia indica DNA containing 0.5 ng, 0.1 ng or 0.01 ng DNA.

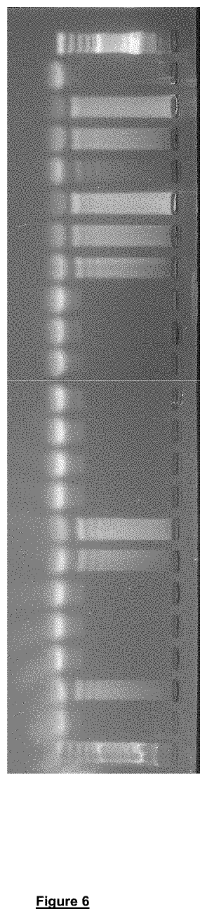

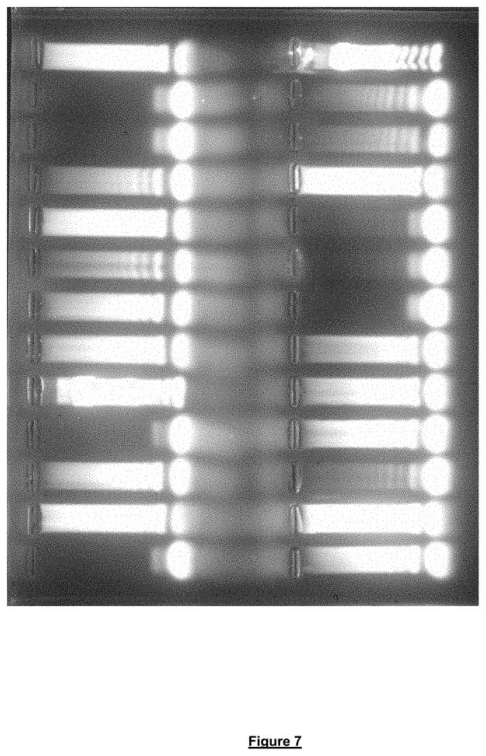

The results obtained with lyophilized mixes after storage at room temperature at day 1, week 1, week 2, week 4 and week 8 were similar to those obtained using fresh mixes at the respective time intervals. At week 12, the results obtained using the lyophilised reagent mixture were not as strong as those obtained with fresh reagent mixture, which may indicate degradation of some the reagent at this time point. The sensitivities also dropped to 0.1 ng of Tilletia indica DNA, as indicated in FIG. 6 (presented in Example 4 below).

### Example 3—Determination of Optimized Protocol for Extraction of DNA from Fungal Spores

In this example, two methods of extracting DNA from fungal spores were compared in an experiment performed at CIMMYT El Batan, Mexico. The purpose of this experiment was to determine the effectiveness of mechanical disruption and freeze-thaw cycles to extract DNA from Karnal bunt fungal spores and the suitability of that DNA for use in the LAMP assay, including the protocol of the disclosure.

DNA extraction was performed according to the following protocols:

### Mechanical Disruption

Twenty μl TE or water was pipetted into a bunted grain, and 1-2 μl was then pipetted onto the centre of a cut 2-3 mm square cut coverslip on a glass slide. Another 2-3 mm square cut coverslip piece was placed over the first piece to make a ‘spores sandwich’. The ‘sandwich’ was verified under the microscope for presence of spores. If present, the sandwich was tapped gently in order to crush the spores and was transferred into a 0.2 ml microtube. A microtip was then used to crush the spore sandwich to fine pieces. A volume of 20 μl TE was added to the microtube and heated at 95° C. for 15 min. A volume of 5 μl of spore extract solution was used in a 25 μl LAMP reaction mixture. Three replicates were performed (designated A1, A2, A3 respectively). Furthermore, duplicate assays were performed for each replicate (designated A11 and A12, A21 and A22, A31 and A32).

### Freeze-Thaw Cycles

A volume of 60-200 μl TE or water was pipetted onto a bunted grain or the pellet obtained from a size-selective sieve wash test [as described in Section 3.2 of International Standards for Phytosanitary Measures Diagnostic Protocol for T. indica, ISPM 27 (2014)]. The volume of TE or water pipetted was dependent on the severity of the bunt or the size of the pellet. The tube was immersed in an ice-salt bath for 5-10 minutes and then immediately placed in a boiling water bath or heating block for a further 5 minutes to thaw. This ‘freeze-thaw’ cycle was repeated three times to produce a spore extract solution. A 5 μl volume of the spore extract solution was used in a 25 μl LAMP reagent mixture. Three replicates were performed (designated B1, B2, B3 respectively). Furthermore, duplicate assays were performed for each replicate (designated B11 and B12, B21 and B22, B31 and B32)

### LAMP Assays

LAMP assays were then performed using the DNA extracted from the Karnal bunt spores, as well as using negative and positive controls. Briefly, a LAMP reagent mixture sufficient for 20 reactions was prepared in accordance with Table 2 of Example 1 herein. 20 μl of LAMP reagent mixture was then aliquoted into 20 PCR tubes. 5 μl of template DNA, negative control DNA or water as a non-template control (NTC) was then added to each tube as set out below:

| Tube # | Sample |
|---|---|
| Tube 1. | Non-template control (NTC) |
| Tube 2. | MKTi8 (0.5 ng) dried |
| Tube 3. | MKTi8 (0.1 ng) dried |
| Tube 4. | MKTi8 (0.01 ng) dried |
| Tube 5. | MKTi8 (0.5 ng) dried |
| Tube 6. | MKTi8 (0.1 ng) dried |
| Tube 7. | MKTi8 (0.01 ng) dried |
| Tube 8. | Negative control F. graminearum |
| Tube 9. | A11 |
| Tube 10. | A12 |
| Tube 11. | A21 |
| Tube 12. | A22 |
| Tube 13. | A31 |
| Tube 14. | A32 |
| Tube 15. | B11 |
| Tube 16. | B12 |
| Tube 17. | B21 |
| Tube 18. | B22 |
| Tube 19. | B31 |
| Tube 20. | B32 |

The PCR tubes were sealed and placed on a BIORAD real-time PCR machine and incubated at 64° C. for 30 minutes and then at 80° C. for 10 min to denature the DNA polymerase.

The LAMP assay reactions were then loaded onto a 2% agarose gel in the following order (from left to right): 100 bp ladder marker; NTC; MKTi8 (0.5 ng); MKTi8 (0.1 ng); MKTi8 (0.01 ng); MKTi8 (0.5 ng); MKTi8 (0.1 ng); MKTi8 (0.01 ng); Negative control F. graminearum; A11; A12; A21; A22; A31; A32; B11; B12; B21; B22; B31; B32; NTC; and 100 bp ladder marker. The LAMP assay amplicons were then resolved by electrophoresis at 90 volts for 70 minutes, the results of which are presented in FIG. 6.

As will be apparent from FIG. 6, Karnal bunt spore solutions that did not undergo a freeze-thaw process (i.e., those that underwent mechanical disruption only) did not result in a LAMP product. On the contrary, a number of those that did under a freeze-thaw process resulted in LAMP products in indicating that the freeze-thaw method is sufficient to break the spore wall and release fungal DNA for analysis and identification. These results suggest that the freeze-thaw method is an effective method for releasing template DNA from fungal spores, and that the resulting DNA is suitable for use in a LAMP assay for diagnosis of Karnal bunt thereby avoiding the need for a commercial DNA extraction kit. On the contrary, DNA extraction from spores by mechanical disruption using a ‘spore sandwich’ (the other method of DNA template preparation endorsed in the ISPM 27 Protocol) was shown to be less effective.

The ability to rely on the freeze-thaw method has the potential to provide significant saving in terms of both cost and time when performing a diagnostic test for Karnal bunt. Furthermore, the ability to rely on the freeze-thaw method also bypasses the rate-limiting germination step required to obtain mycelium for DNA extraction for standard PCR diagnosis, one of the methods endorsed in the International Standards for Phytosanitary Measures Diagnostic Protocol for T. indica, ISPM 27.

### Example 4—Sensitivity of Detection by LAMP Assay of Karnal Bunt DNA Extracted Using the Freeze-Thaw Method

In this example, the inventors assess the ability to detect different amounts of Karnal bunt DNA extracted using the freeze-thaw method using the LAMP assay.

The Karnal bunt DNA tested in this experiment was obtained as follows:

### Bunted Grain Sample 1

DNA was extracted from a first bunted grain designated ‘Bunted Grain Sample 1’. Briefly, 200 μl of water was added to a 1.5 ml micro-tube containing the bunted grain (TE would work equally well). The tube was then flicked to release as many spores as possible into solution. 200 μl of the spore-containing solution was then transferred to a fresh 1.5 ml micro-tube and mixed by pipetting to ensure a homogenous spore solution. Aliquots of varying quantities of the spore-containing solution were then transferred to 4 fresh micro-tubes as follows: 60 μl (B1); 40 μl (B2), 30 μl (B3) and 20 μl (B4). To release Tilletia indica DNA from the spores, the spores containing solutions B1-B4 were then subjected to three freeze-thaw cycles by immersion in an ice-salt bath followed by heating in a boiling water bath as described in Example 3. An aliquot was then counted in a haemocytometer.

### Bunted Grain Sample 2

DNA was then extracted from a further bunted grain designated ‘Bunted Grain Sample 2’. As described above for Sample 1, 200 μl of water was added to a 1.5 ml micro-tube containing the bunted grain. The tube was then flicked to release as many spores as possible into solution. This particular grain was very bunted resulting in a spore solution which appeared concentrated. As such, a further 200 μl of water was added to the micro-tube to provide a total spore-containing solution volume of 400 μl. The 400 μl of spore-containing solution was then transferred to a fresh 1.5 ml micro-tube and mixed by pipetting to ensure a homogenous spore solution. Aliquots of varying quantities of the spore-containing solution were then transferred to 4 fresh micro-tubes as follows: 60 μl (B5); 40 μl (B6), 30 μl (B7) and 20 μl (B8). To release Tilletia indica DNA from the spores, the spore-containing solutions B5-B8 were then subjected to three freeze-thaw cycles by immersion in an ice-salt bath for 5-10 minutes followed by a thawing on a dry heat block at 99 degree Celcius for 5 minutes. An aliquot was counted in a haemocytometer.

Tilletia foetida Negative Control

DNA was then extracted from a grain infected by Tilletia foetida for use as a negative control. Briefly, 100 μl water was added to a 1.5 ml micro-tube containing a grain bunted with Tilletia foetida. The tube was then flicked to release as many spores as possible into solution. 20 μl of spore-containing solution was then transferred to a fresh 1.5 ml micro-tube and mixed by pipetting to ensure a homogenous spore solution. To release Tilletia foetida DNA from the spores, the spore-containing solution was then subjected to three freeze-thaw cycles by immersion in an ice-salt bath for 5-10 minutes followed by heating in a boiling water bath as described in Example 3.

Tilletia indica Spores (Ti)

A positive control was also prepared. Spore-containing solutions were prepared from grain bunted with Tilletia indica as described above. Spores were isolated from the spore-containing solution using a nylon mesh, 15 micron pores (spectrum labs.com) wetted with 3% KOH solution. Five spores were picked from the nylon mesh and placed into 5 μl TE pH 8.0 in a micro-tube. To release Tilletia indica DNA from the spores, the micro-tube containing the spore solution was then subjected to three freeze-thaw cycles by immersion in an ice-salt bath for 5-10 minutes followed by heating in a boiling water bath as described in Example 3.

### LAMP Assays

LAMP assays were then performed using the DNA extracted from the samples above. Briefly, a LAMP reagent mixture sufficient for 25 reactions was prepared in accordance with Table 2 of Example 1 herein. 20 μl of LAMP reagent mixture was then aliquoted into 22 PCR tubes. 5 μl of template DNA, negative control DNA or water as a non-template control (NTC) was then added to each tube as set out below:

| Tube # | Sample |
|---|---|
| Tube 1. | Non-template control (NTC) |
| Tube 2. | MKTi8 (0.5 ng) dried |
| Tube 3. | MKTi8 (0.1 ng) dried |
| Tube 4. | T. foeitda negative control |
| Tube 5. | B11 |
| Tube 6. | B12 |
| Tube 7. | B21 |
| Tube 8. | B22 |
| Tube 9. | B31 |
| Tube 10. | B32 |
| Tube 11. | B41 |
| Tube 12. | B42 |
| Tube 13. | B51 |
| Tube 14. | B52 |
| Tube 15. | B61 |
| Tube 16. | B62 |
| Tube 17. | B71 |
| Tube 18. | B72 |
| Tube 19. | B81 |
| Tube 20. | B82 |
| Tube 21. | Ti (DNA from 5 Ti spores) |
| Tube 22. | MKTi8 (0.1 ng) dried |
| Tube 23. | MKTi8 (0.5 ng) dried (with Freeze dried MM) |
| Tube 24. | MKTi8 (0.1 ng) dried (with Freeze-dried MM) |

The PCR tubes were sealed and placed on a BIORAD real-time PCR machine and incubated at 64° C. for 30 minutes and then at 80° C. for 10 min to denature the DNA polymerase.

The LAMP assay reactions from this example were evaluated on an agarose gel together with the LAMP assay reactions for MKTi8 (0.5 ng and 0.1 ng) performed using freeze-dried master-mix in Example 2 and the LAMP assay reactions for MKTi8 (0.5 ng/μl, 0.1 ng and 0.01 ng) performed in Example 3. The respective LAMP assay reactions were loaded onto a 2% agarose gel in the following order:

Top Row of Gel (from Left to Right):

NTC (Example 4); MKTi8 (0.5 ng) (Example 3); MKTi8 (0.1 ng) (Example 3); Tfoeitda negative control (Example 4); 100 bp ladder marker; B11 (Example 4); B12 (Example 4); B21 (Example 4); B22 (Example 4); B31 (Example 4); B32 (Example 4); B41 (Example 4); and B42 (Example 4); and

Bottom Row of Gel (from Left to Right):

B51 (Example 4); B52 (Example 4); B61 (Example 4); B62 (Example 4); B71 (Example 4); B72 (Example 4); B81 (Example 4); B82 (Example 4); Ti (DNA from 5 Ti spores) (Example 4); MKTi8 (0.01 ng) (Example 3); MKTi8 (0.5 ng) (Example 2); MKTi8 (0.1 ng) (Example 2); and 100 bp ladder marker.

The LAMP assay amplicons were then resolved by electrophoresis at 90 volts for 70 minutes, the results of which are presented in FIG. 7.

As can be seen, the LAMP assay was able to detect DNA obtained from Tilletia indica spores by the freeze-thaw process with good specificity, even when DNA was obtained from as few as five spores.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The invention may be defined according to one or more of the following statements:

Statement 1. A test kit for detecting Tilletia indica or spores thereof in a sample, said test kit comprising:
(1) a reagent mixture comprising reagents configured to amplify a nucleic acid sequence of Tilletia indica by an isothermal amplification assay to produce a biotinylated amplification product comprising a sequence set forth in SEQ ID NO: 1;
(2) a nucleic acid probe comprising a polynucleotide sequence of at least 16 nucleotides in length which is sufficiently complementary to a region of corresponding length within the biotinylated amplification product such that the nucleic acid probe and amplification product are hybridisable, wherein the nucleic acid probe is conjugated to a hapten;
(3) one or more lateral flow test strips comprising
   (a) a label-holding portion comprising a mobilisable capture reagent comprising a detectable label, wherein the mobilisable capture reagent is configured to bind to the hapten conjugated to the nucleic acid probe; and
   (b) a test portion comprising an immobilised capture reagent configured to specifically bind biotin and thereby immobilise biotin to the test portion.
      Statement 2. The test kit according to statement 1, wherein the reagent mixture comprises:
   at least one DNA polymerase enzyme;
   isothermal amplification primers specific for Tilletia indica;
   dinucleotide triphosphates (dNTPs);
   one or more salts; and
   a buffer.
      Statement 3. The test kit according to statement 1 or 2, wherein the isothermal amplification assay is selected from loop-mediated isothermal amplification (LAMP), recombinase polymerase amplification (RPA) and helicase-dependent amplification (HDA).
      Statement 4. The test kit according to statement 1 or 2, wherein the isothermal amplification assay is a LAMP assay.
      Statement 5. The test kit according to statement 4, wherein the reagent mixture comprises:
   at least one DNA polymerase enzyme;
   LAMP primers specific for Tilletia indica;
   dinucleotide triphosphates (dNTPs);
   a magnesium salt; and
   a buffer.
      Statement 6. The test kit according to statement 5, wherein the reagent mixture comprises the following LAMP primers:

Ti-FIP comprising the sequence set forth in SEQ ID NO:2 or a sequence which is substantially identical thereto, wherein the sequence of Ti-FIP is conjugated to a biotin;

Ti-BIP comprising the sequence set forth in SEQ ID NO:3 or a sequence which is substantially identical thereto;

Ti-LF comprising the sequence set forth in SEQ ID NO:4 or a sequence which is substantially identical thereto;

Ti-LB comprising the sequence set forth in SEQ ID NO:5 or a sequence which is substantially identical thereto;

Ti-F3 comprising the sequence set forth in SEQ ID NO:6 or a sequence which is substantially identical thereto; and

Ti-B3 comprising the sequence set forth in SEQ ID NO:7 or a sequence which is substantially identical thereto.

Statement 7. The test kit according to statement 6, wherein the LAMP primers are:

Ti-FIP (SEQ ID NO:2) conjugated to a biotin;

Ti-BIP (SEQ ID NO:3);

Ti-LF (SEQ ID NO: 4);

Ti-LB (SEQ ID NO: 5);

Ti-F3 (SEQ ID NO: 6); and

Ti-B3 (SEQ ID NO: 7).

Statement 8. The test kit according to any one of statements 5 to 7, wherein the DNA polymerase enzyme is selected from the groups consisting of Bst DNA polymerase, Bsm DNA polymerase, Gst DNA polymerase, SD DNA polymerase and combinations thereof.
Statement 9. The test kit according to statement 8, wherein the DNA polymerase enzyme is Bst DNA polymerase.
Statement 10. The test kit according to any one of statements 5 to 9, wherein the magnesium salt is MgSO₄.
Statement 11. The test kit according to any one of statements 1 to 10, wherein the reagent mixture is thermostable.
Statement 12. The test kit according to any one of statements 1 to 11, wherein the reagent mixture and the nucleic acid probe are each provided in a dried form.
Statement 13. The test kit according to statement 11 or 12, wherein the reagent mixture comprises a cryoprotectant.
Statement 14. The test kit according to statement 13, wherein the cryoprotectant is sucrose or trehalose.
Statement 15. The test kit according to statement 13 or 14, wherein the cryoprotectant is present at about 7% w/v to about 8% w/v.
Statement 16. The test kit according to any one of statements 1 to 10, wherein the reagent mixture is provided in a liquid or frozen form and the nucleic acid probe is provided in a dried form.
Statement 17. The test kit according to any one of statement 1 to 16, wherein the nucleic acid probe is substantially identical to the polynucleotide sequence set forth in SEQ ID NO:8.
Statement 18. The test kit according to any one of statement 1 to 17, wherein the nucleic acid probe comprises the polynucleotide sequence set forth in SEQ ID NO: 8.
Statement 19. The test kit according to any one of statements 1 to 15, wherein:
(a) the reagent mixture is thermostable and comprises:
(i) a Bst DNA polymerase
(ii) the LAMP primers: Ti-FIP (SEQ ID NO: 2) conjugated to a biotin; Ti-BIP (SEQ ID NO: 3); Ti-LF (SEQ ID NO: 4); Ti-LB (SEQ ID NO: 5); Ti-F3 (SEQ ID NO: 6); and Ti-B3 (SEQ ID NO: 7);
(iii) dNTPs;
(iv) MgSO₄; and
(v) a buffer; and
   (b) the nucleic acid probe comprises the polynucleotide sequence set forth in SEQ ID NO: 8.
   Statement 20. The test kit according to any one of statements 1 to 19, wherein the immobilised capture reagent is a biotin ligand.
   Statement 21. The test kit according to any one of statements 1 to 20, wherein the mobilisable capture reagent of the label-holding portion is an antibody which binds the hapten, and wherein the antibody is conjugated to the detectable label.
   Statement 22. The test kit according to any one of statements 1 to 21, wherein the hapten is a fluorophore selected from FITC or FAM, or digoxigenin.
   Statement 23. The test kit according to statement 22, wherein the hapten is FITC.
   Statement 24. The test kit according to any one of statements 1 to 23, wherein the detectable label is a gold nanoparticle, latex nanoparticle or a fluorescent quantum dot (QD).
   Statement 25. The test kit according to any one of statements 1 to 24, wherein the one or more test strips comprise a control portion comprising an immobilised capture reagent configured to detect the mobilisable capture reagent.
   Statement 26. The test kit according to statement 25, wherein immobilised capture reagent at the control portion is an antibody against the mobilisable capture reagent.
   Statement 27. The test kit according to statement 25 or 26, wherein the control portion is located downstream of the test portion on the test strip.
   Statement 28. The test kit according to any one of statements 1 to 27, wherein the one or more test strips comprises a sample receiving portion for receiving a liquid sample.
   Statement 29. The test kit according to any one of statements 1 to 28, further comprising an aqueous buffer for the test strip(s).
   Statement 30. The test kit according to any one of statements 1 to 29, further comprising an assay positive control comprising isolated Tilletia indica DNA or a fragment thereof suitable as a template for the isothermal amplification assay.
   Statement 31. The test kit according to any one of statements 1 to 30, comprising an apparatus configured to receive the test strip(s) and present information about the identification of Tilletia indica or spores thereof in a sample to a user via a display.
   Statement 32. The test kit according to statement 31, wherein the apparatus is provided in the form of a hand-held device.
   Statement 33. The test kit according to statement 31 or 32, wherein the apparatus comprises a reader to identify Tilletia indica or spores thereof in the sample.
   Statement 34. The test kit according to statement 33, wherein the reader includes one or more photodetectors capable of monitoring light reflection or light output at the test portion.
   Statement 35. A method of detecting the presence or absence of Tilletia indica nucleic acids in a sample, said method comprising:
(a) performing an isothermal amplification assay on DNA extracted from the sample (sample DNA) using a reagent mixture of the test kit according to any one of statements 1 to 34;
(b) incubating the product of (a) in the presence of a nucleic acid probe of the test kit according to any one of statements 1 to 34;
(c) contacting the product of (b) with the sample receiving portion of a test strip of the test kit according to any one of statements 1 to 34;
(d) detecting the presence or absence of an amplification product at the test portion of the test strip; and
(e) determining the presence or absence of Tilletia indica nucleic acids in the sample based on the presence or absence of an amplification product at the test portion.
   Statement 36. The method according to statement 35, wherein:
(i) detecting a signal at the test portion is indicative of the presence of Tilletia indica nucleic acids in the sample; and
(ii) detecting no signal at the test portion is indicative of the absence of Tilletia indica nucleic acids in the sample.
   Statement 37. The method according to statement 35 or 36, wherein the detecting a signal at the control portion of the test strip is indicative that the sample has flowed through the test strip.
   Statement 38. The method according to any one of statements 35 to 37, wherein a volume of buffer is added to the sample following the incubation at (b) and prior to contacting the sample with the sample receiving portion of the test strip.
   Statement 39. The method according to any one of statements 35 to 38, comprising the step of isolating DNA from the sample prior to step (a).
   Statement 40. The method according to any one of statements 35 to 39, wherein the reagent mixture is provided in a dried form and water is added to thermostable reagent mixture at (a).
   Statement 41. The method according to any one of statements 35 to 40, wherein the isothermal amplification assay is performed at about 60° C. to about 66° C.
   Statement 42. The method according to any one of statements 35 to 41, wherein the isothermal amplification assay is a loop-mediated isothermal amplification assay (LAMP).
   Statement 43. The method according to any one of statements 35 to 42, wherein the incubation step at (b) is performed at about 64° C.
   Statement 44. The method according to any one of statements 35 to 43, wherein the sample is grain or a part thereof, a commodity comprising grain or a part thereof, or debris comprising grain or a part thereof as a contaminant.
   Statement 45. The method according to statement 44, wherein the grain is from wheat or triticale.

1. A test kit for detecting Tilletia indica or spores thereof in a sample, said test kit comprising:
(1) a reagent mixture comprising reagents configured to amplify a nucleic acid sequence of Tilletia indica by an isothermal amplification assay to produce a biotinylated amplification product comprising a sequence set forth in SEQ ID NO: 1;
(2) a nucleic acid probe comprising a polynucleotide sequence of at least 16 nucleotides in length which is sufficiently complementary to a region of corresponding length within the biotinylated amplification product such that the nucleic acid probe and amplification product are hybridisable, wherein the nucleic acid probe is conjugated to a hapten;
(3) one or more lateral flow test strips comprising
(a) a label-holding portion comprising a mobilisable capture reagent comprising a detectable label, wherein the mobilisable capture reagent is configured to bind to the hapten conjugated to the nucleic acid probe; and
(b) a test portion comprising an immobilised capture reagent configured to specifically bind biotin and thereby immobilise biotin to the test portion.

2. The test kit according to claim 1, wherein the reagent mixture comprises:
at least one DNA polymerase enzyme;
isothermal amplification primers specific for Tilletia indica;
dinucleotide triphosphates (dNTPs);
one or more salts; and
a buffer.

3. The test kit according to claim 1, wherein the isothermal amplification assay is selected from loop-mediated isothermal amplification (LAMP), recombinase polymerase amplification (RPA) and helicase-dependent amplification (HDA).

4. The test kit according to claim 1, wherein the isothermal amplification assay is a LAMP assay.

5. The test kit according to claim 4, wherein the reagent mixture comprises:
at least one DNA polymerase enzyme;
LAMP primers specific for Tilletia indica;
dinucleotide triphosphates (dNTPs);
a magnesium salt; and
a buffer.

6. The test kit according to claim 5, wherein the reagent mixture comprises the following LAMP primers:
Ti-FIP comprising the sequence set forth in SEQ ID NO:2 or a sequence which is substantially identical thereto, wherein the sequence of Ti-FIP is conjugated to a biotin;
Ti-BIP comprising the sequence set forth in SEQ ID NO:3 or a sequence which is substantially identical thereto;
Ti-LF comprising the sequence set forth in SEQ ID NO:4 or a sequence which is substantially identical thereto;
Ti-LB comprising the sequence set forth in SEQ ID NO:5 or a sequence which is substantially identical thereto;
Ti-F3 comprising the sequence set forth in SEQ ID NO:6 or a sequence which is substantially identical thereto; and
Ti-B3 comprising the sequence set forth in SEQ ID NO:7 or a sequence which is substantially identical thereto.

7. The test kit according to claim 6, wherein the LAMP primers are:
Ti-FIP (SEQ ID NO:2) conjugated to a biotin;
Ti-BIP (SEQ ID NO:3);
Ti-LF (SEQ ID NO: 4);
Ti-LB (SEQ ID NO: 5);
Ti-F3 (SEQ ID NO: 6); and
Ti-B3 (SEQ ID NO: 7).

8. The test kit according to claim 5, wherein
(i) the DNA polymerase enzyme is selected from the groups consisting of Bst DNA polymerase, Bsm DNA polymerase, Gst DNA polymerase, SD DNA polymerase and combinations thereof; and/or
(ii) the magnesium salt is MgSO₄.

9. The test kit according to claim 1, wherein:
(i) the reagent mixture is thermostable; and/or
(ii) the reagent mixture and the nucleic acid probe are each provided in a dried form.

10. The test kit according to claim 9, wherein the reagent mixture comprises a cryoprotectant.

11. The test kit according to claim 10, wherein:
(i) the cryoprotectant is sucrose or trehalose; and/or
(ii) the cryoprotectant is present at about 7% w/v to about 8% w/v.

12. The test kit according to claim 1, wherein the reagent mixture is provided in a liquid or frozen form and the nucleic acid probe is provided in a dried form.

13. The test kit according to claim 1, wherein the nucleic acid probe is substantially identical to the polynucleotide sequence set forth in SEQ ID NO:8 or comprises the polynucleotide sequence set forth in SEQ ID NO: 8.

14. The test kit according to claim 1, wherein:
(a) the reagent mixture is thermostable and comprises:
(i) a Bst DNA polymerase
(ii) the LAMP primers: Ti-FIP (SEQ ID NO: 2) conjugated to a biotin; Ti-BIP (SEQ ID NO: 3); Ti-LF (SEQ ID NO: 4); Ti-LB (SEQ ID NO: 5); Ti-F3 (SEQ ID NO: 6); and Ti-B3 (SEQ ID NO: 7);
(iii) dNTPs;
(iv) MgSO₄; and
(v) a buffer; and
(b) the nucleic acid probe comprises the polynucleotide sequence set forth in SEQ ID NO: 8.

15. The test kit according to claim 1, wherein:
(i) the immobilised capture reagent is a biotin ligand;
(ii) the mobilisable capture reagent of the label-holding portion is an antibody which binds the hapten, and wherein the antibody is conjugated to the detectable label;
(iii) the hapten is a fluorophore selected from FITC or FAM, or digoxigenin;
(iv) the detectable label is a gold nanoparticle, latex nanoparticle or a fluorescent quantum dot (QD);
(iv) the one or more test strips comprise a control portion comprising an immobilised capture reagent configured to detect the mobilisable capture reagent
(v) the one or more test strips comprises a sample receiving portion for receiving a liquid sample.

16. The test kit according to claim 15, wherein:
(i) the immobilised capture reagent at the control portion is an antibody against the mobilisable capture reagent; and/or
(ii) the control portion is located downstream of the test portion on the test strip.

17. The test kit according to claim 1, comprising:
(i) an aqueous buffer for the test strip(s);
(ii) an assay positive control comprising isolated Tilletia indica DNA or a fragment thereof suitable as a template for the isothermal amplification assay;
(iii) an apparatus configured to receive the test strip(s) and present information about the identification of Tilletia indica or spores thereof in a sample to a user via a display.

18. The test kit according to claim 17, wherein:
(i) the apparatus is provided in the form of a hand-held device; and/or
(ii) the apparatus comprises a reader to identify Tilletia indica or spores thereof in the sample.

19. The test kit according to claim 18, wherein the reader includes one or more photodetectors capable of monitoring light reflection or light output at the test portion.

20. A method of detecting the presence or absence of Tilletia indica nucleic acids in a sample, said method comprising:
(a) performing an isothermal amplification assay on DNA extracted from the sample (sample DNA) using a reagent mixture of the test kit according to claim 1;
(b) incubating the product of (a) in the presence of a nucleic acid probe of the test kit according to claim 1;
(c) contacting the product of (b) with the sample receiving portion of a test strip of the test kit according to claim 1;
(d) detecting the presence or absence of an amplification product at the test portion of the test strip; and
(e) determining the presence or absence of Tilletia indica nucleic acids in the sample based on the presence or absence of an amplification product at the test portion.

21. The method according to claim 20, wherein:
(i) detecting a signal at the test portion is indicative of the presence of Tilletia indica nucleic acids in the sample; and
(ii) detecting no signal at the test portion is indicative of the absence of Tilletia indica nucleic acids in the sample.

22. The method according to claim 20, wherein one or more of the following applies:
(i) the detecting a signal at the control portion of the test strip is indicative that the sample has flowed through the test strip;
(ii) a volume of buffer is added to the sample following the incubation at (b) and prior to contacting the sample with the sample receiving portion of the test strip;
(iii) the reagent mixture is provided in a dried form and water is added to thermostable reagent mixture at (a);
(iv) the isothermal amplification assay is performed at about 60° C. to about 66° C.;
(v) the isothermal amplification assay is a loop-mediated isothermal amplification assay (LAMP);
(vi) the incubation step at (b) is performed at about 64° C.; and/or
(vii) the sample is grain or a part thereof, a commodity comprising grain or a part thereof, or debris comprising grain or a part thereof as a contaminant.

23. The method according to claim 22, wherein the grain is from wheat or triticale.
